(19) United States
(12) Patent Application Publication
(10) Pub. No.: US 2026/0290540 A1
(43) Pub. Date: Sep. 24, 2026

(54) SYSTEM AND METHOD FOR CREATING A QUANTIFIABLE IVF PHENOTYPE MAP TO DRIVE DISCOVERY IN IVF PROGNOSTICS

(71) Applicant: Univfy Inc., Los Altos, CA (US)

(72) Inventor: Mylene YAO, Los Altos, CA (US)

(21) Appl. No.: 19/472,435

(22) PCT Filed: Apr. 5, 2024
(86) PCT No.: PCT/US2024/023260

§ 371 Date: Oct. 3, 2025

Related U.S. Application Data
(60) Provisional application No. 63/494,441, filed on Apr. 5, 2023.

(51) Int. Cl. G16H 20/40; G16B 20/20; G16H 50/30

(52) U.S. Cl. CPC G16H 20/40; G16B 20/20; G16H 50/30

(57) ABSTRACT

Provided herein are computer-implemented methods for determining the probability of a patient having an IVF failure. Also provided are computer-implemented methods for predicting the probability of the patient having an IVF phenotype or a recurring IVF phenotype. Also provided are computer-implemented methods for predicting the probability of the patient having an intermediate IVF treatment outcome.

### CROSS-REFERENCES

This application claims priority to U.S. Provisional Application No. 63/494,441, filed Apr. 5, 2023, the contents of which are herein incorporated by reference in its entirety.

### BACKGROUND

Establishing equitable access to safe and effective fertility treatments including in vitro fertilization (IVF), assisted reproductive technology (ART), and novel therapeutics, has the most immediate and transformative impact on an estimated 10 million families in the US and over 100 million families globally that need fertility care to have a baby. IVF is the safest and most effective treatment for clinical infertility experienced by one in six couples, LGBTQ couples and single women, as well as people seeking fertility preservation for medical or ovarian aging reasons. Barriers to IVF are complex, with emotional stress, uncertainty of treatment success, and financial cost exacerbated by inadequate insurance coverage or lack thereof often cited as the top reasons for its underutilization.

Despite over four decades of using IVF treatment producing over 8 million babies worldwide, IVF and related assisted reproductive technologies are still perceived as “unpredictable” and financially risky as without guarantee of having a baby. IVF failure and especially recurrent IVF failure or unexpected embryology outcomes are emotionally upsetting as providers often do not have an explanation and there is a paucity of information to support whether there is a high risk of recurrent IVF failure or embryology outcomes associated with poor prognosis. Conversely, it is often unclear if IVF failure that is concurrent with good embryology outcomes is associated with good prognosis for the next IVF treatment. Currently, the only way for most patients to know if IVF is a good option is to persevere to do several IVF treatments, which often require significant financial resources, emotional cost, and physical intervention. It would be desirable to spare patients from the financial, emotional and physical costs of IVF if IVF would not give them a baby. Also, the ability to identify patients whose needs are not met by IVF or other available treatments can inform and accelerate the development of new therapeutics to help these patients have a family. As importantly, for most patients, learning that IVF is an excellent treatment option will give further reassurance to improve their confidence to start IVF treatment sooner.

Accordingly, there is a need for improved methods for determining the probability of a female patient to have an IVF treatment failure, a recurrent IVF treatment failure, or recurrent poor intermediate IVF treatment outcomes.

### SUMMARY

In one aspect, provided herein is a computer-implemented method comprising: (a) inputting, into a first model (a predictive model), an IVF dataset from at least one group of unique patient-treatment incidences at one or more treatment centers, wherein the IVF dataset comprises (i) data generating at least one IVF phenotype assignment for each of the unique patient-treatment incidences, wherein each IVF phenotype assignment is a categorical variable or a numerical variable, (i) IVF outcomes data, (ii) intermediate IVF treatment outcomes data, and (iv) clinical data, wherein the IVF outcomes data comprises at least one variable of an IVF outcome, wherein an IVF outcomes variable is an IVF outcomes failure variable, an IVF treatment outcomes variable is an IVF outcomes success variable, or the IVF outcomes variable is an IVF outcomes pending variable, wherein the intermediate IVF treatment outcomes data comprises a plurality of variables of one or more intermediate IVF treatment outcomes selected from the group consisting of patient oocyte status variables, patient embryo status variables, carrier status variables, and any combination thereof, wherein the clinical data comprises at least one clinical variable, wherein the at least one IVF phenotype assignment variable for each unique patient-treatment incidence is based on the at least one intermediate IVF treatment outcomes data variable and the at least one clinical variable; and (b) training the first model using the at least one IVF phenotype assignment and an IVF outcome variable for each of the unique patient-treatment incidences, thereby generating trained first model outputs for the unique patient-treatment incidences.

In some embodiments, the method further comprises: (c) applying, to the trained first model, a target's patient data comprising at least one intermediate IVF treatment variable, at least one clinical variable, and at least one IVF phenotype assigned to the target patient; and (d) generating a trained first model output for the target patient selected from the group consisting of the target patient's probability of IVF failure, the target patient's probability of at least one occurring or recurring IVF phenotype, and a combination thereof.

In another aspect, provided herein is a computer-implemented method comprising: (a) inputting, into a first model, an IVF dataset from at least one group of unique patient-treatment incidences at one or more treatment centers, wherein the IVF dataset comprises (i) data enabling generation of at least one concordance model output of a value of greater or more than expected, same as expected, or fewer or less than expected for each of the unique patient-treatment, (ii) IVF outcomes data, (iii) intermediate IVF treatment outcomes data, and (iv) clinical data; wherein the IVF outcomes data comprises at least one variable of an IVF treatment outcome, wherein an IVF outcomes variable is an IVF outcomes failure variable, an IVF treatment outcomes variable is an IVF outcomes success variable, or an IVF outcomes variable is an IVF outcomes pending variable; wherein the intermediate IVF treatment outcomes data comprise a plurality of variables of one or more intermediate IVF treatment outcomes selected from the group consisting of patient oocyte status variables, patient embryo status variables, carrier status variables, and any combination thereof; wherein the clinical data comprises at least one clinical variable; (b) generating a concordance model output for each of the unique patient-treatment incidences comprising: (i) inputting, into an untrained concordance model, at least one first concordance input and at least one second concordance input, wherein each first concordance input comprises at least one intermediate IVF treatment outcomes variable or clinical variable; wherein each second concordance input comprises at least one intermediate IVF treatment outcomes or clinical variable; (ii) training the concordance model to generate a concordance model output; and (iii) outputting, from the trained concordance model, the concordance model output for each of the unique patient-treatment incidences; (c) training the first model to generate trained first model outputs using at least one concordance model output for each of the unique patient-treatment incidences; wherein the trained first model output comprises one or more selected from the group consisting of (i) a probability of IVF failure, (ii) a probability of at least one occurring or recurring IVF phenotype, and (iii) a probability of at least one occurring or recurring concordance model output.

In some embodiments, the method further comprises: (d) applying, to the trained concordance model, a target patient's data comprising (i) the target patient's first concordance input comprising at least one intermediate IVF treatment outcomes variable or clinical variable and (ii) the target patient's second concordance input comprising at least one intermediate IVF treatment outcomes variable or clinical variable, wherein the at least one intermediate IVF treatment outcomes variable or clinical variable are not identical in the target patient's first and second concordance inputs; and (e) generating the target patient's concordance model output; (f) applying, into the trained first model, the target patient's concordance model output; and (g) generating a first model output for the target patient comprising one or more selected from the group consisting of (i) the target patient's probability of IVF failure, (ii) the target patient's probability of at least one occurring or recurring IVF phenotype, and (iii) the target patient's probability of at least one occurring or recurring concordance model output.

In some embodiments of any of the methods provided, the at least one of the IVF outcomes failure variables is selected from the group consisting of: no pregnancy; an undetectable serum B-HCG level of the target patient by 21 days after embryo transfer; a biochemical pregnancy loss after an initially detectable and positive serum B-HCG level declines or lowers to an undetectable levels; a disappearance of a gestational sac; an arrest in fetal development after an earlier detectable stage of fetal development; a diagnosis of a molar pregnancy, and a diagnosis of an ectopic pregnancy.

In some embodiments of any of the methods provided, the IVF outcomes success variable is a live birth.

In some embodiments of any of the methods provided, the at least one IVF outcomes pending variable is selected from the group consisting of (i) at least one planned, intended or contemplated IVF procedure selected from the group consisting of an oocyte retrieval, an in vitro fertilization, an intracytoplasmic sperm injection (ICSI), an embryo culture, an embryo transfer, and an embryo cryopreservation; (ii) occurrence of an embryo transfer but a serum B-HCG test result for the test patient is not available; (iii) at least one planned test selected from the group consisting of a serum B-HCG test, an ultrasound or other imaging test for a gestational sac, an ultrasound or other imaging test for a fetal pole, and an ultrasound or other imaging test for a fetal cardiac activity; (iv) at least one cryopreserved embryo available for transfer; and (v) a variable that is not a variable of the IVF outcome failure or the IVF outcome success

In some embodiments of any of the methods provided, the patient oocyte status variables are selected from the group consisting of: a cancelled oocyte retrieval procedure due to an inadequate ovarian follicular response based on any criteria (e.g., the number of follicles reaching a certain size and/or serum estradiol levels); an oocyte retrieval procedure resulting in fewer than expected number of oocytes or no oocytes; an occurrence of premature ovulation; signs or symptoms indicative of ovarian hyperstimulation syndrome (OHSS); 20% or more retrieved oocytes show abnormal morphology or morphology indicative of development arrest such as intact germinal vesicle, immature oocytes, fragmentation, vesicular appearance, granularity, or any other abnormality present in 20% or more of affected oocytes; 20% or more retrieved oocytes have zona pellucida that is thin, thick or absent; and 20% or more retrieved oocytes have abnormalities.

In some embodiments of any of the methods provided, the patient embryo status variables are selected from the group consisting of: a fewer than 2 blastocysts, or euploid blastocysts if preimplantation genetic testing for aneuploidy is performed; at least 20% of embryos at each developmental stage failing to progress to the next developmental stage; at least 20% of blastocysts do not show expansion by day 5, 6, or 7; at least 20% of blastocysts not obtaining a Grade5AA score based on the Gardner score; 20% or more retrieved oocytes failing to develop into mature oocytes, not achieving the 2PN status after in vitro fertilization, not achieving 2PN after ICSI procedure or attempt; at least 20% of blastocysts are not eligible for cryopreservation or uterine transfer; at least 20% of oocytes and embryos at each developmental stage having one or more detectable abnormal features; at least 20% of oocytes and/or embryos at each developmental stage having an accelerated or delayed timing to progress to the next developmental stage; at least 20% of oocytes and/or embryos having an overall accelerated or delayed progression of two more developmental stages; at least 20% of oocytes and/or embryos being cultured in an abnormal in vitro culture media or environment; at least 20% of embryos at each stage are not progressing to the next stage; at least 20% of oocytes and embryos derived from an abnormal oocyte, sperm, embryo, or an abnormal oocyte-sperm interaction; at least 20% of oocytes and/or embryos having an abnormal level of at least one nucleic acid transcript; at least 20% of oocytes and/or embryos having an abnormal level of at least one polypeptide; at least 20% of oocytes and/or embryos having an abnormal level or structure of at least one intracellular or secreted molecule, analyte, cellular or gene product; and at least 20% of oocytes and/or embryos having at least one gene variant associated with or causes or confers risk for IVF failure, occurrence or recurrence of at least one IVF phenotype, pregnancy loss, recurrent pregnancy loss, fetal developmental defects, congenital birth defects, newborn illnesses.

In some embodiments of any of the methods provided, the carrier status variables are selected from the group consisting of: a less than expected or less than 7 mm thickness of an intended gestational carrier's endometrial lining within 14 days of an embryo transfer; a less than expected or less than 7 mm thickness of an intended gestational carrier's endometrial lining within 48 hours of an embryo transfer; an inadequate thickness of an intended gestational carrier's endometrial lining resulting in a cancelled oocyte retrieval, a cancelled embryo transfer or a freezing of embryos; an endometrium abnormality (e.g., scarring, calcification, polyp(s), cyst(s), nodule(s), or any hypodensity feature); an endometrial cavity fluid or mass; abnormal blood flow in the endometrium, uterine wall, or uterine vasculature; and an abnormal appearance (e.g., an absence of trilaminar appearance) or abnormality of the female reproductive tract.

In some embodiments of any of the methods provided, the at least one clinical variable is selected from the group consisting of: a clinical variable selected from the group consisting of body mass index (BMI), relative distances between any 2 or more facial features, ratio of upper to lower body, relative length of certain body part, abdominal girth, body fat density, and muscle mass of an oocyte source, sperm source, and/or gestational carrier, and qualitative biometrics, quantitative biometrics, and other biometrics of an oocyte source, sperm source, and/or gestational carrier; an ovarian function variable selected from the group consisting of serum anti-mullerian hormone (AMH) levels, day 3 follicle stimulating hormone (D3 FSH) levels, antral follical count (AFC), serum progesterone levels, serum inhibin levels, biochemical analysis or gene expression of granulosa cells or follicular fluid, premature ovarian failure, ovarian failure, medical conditions associated with decreased ovarian function or premature ovarian failure, exposure to medicines or substances harmful to ovarian function; an IVF treatment protocol variable selected from the group consisting of IVF treatment protocol, type of medication, dosage, dosage dose schedule and adjustments, the use of ovarian suppression medication, gonadotropin medication, oral contraceptive pill, gonadotropin releasing hormone (GnRH) agonists, GnRH antagonists, luteinizing hormone (LH), human chorionic gonadotropin (HCG), progesterone or any progestin type, estradiol or any estrogen type, clomiphene, letrozole, recombinant hormones, purified hormones, long-acting, in vitro maturation of oocytes, in vitro gametogenesis; a demographic variable selected from the group consisting of age, race, ethnicity, social determinants of health, health insurance coverage, IVF coverage, household income, education level, type of employment; a reproductive history variable selected from the group consisting of number of months or years of conception attempts, a same-sex couple, a heterosexual couple, a single woman, a history of pregnancy, a number or live birth pregnancy loss; a number of past fertility treatments and outcomes, and a type of past fertility treatments and outcomes; a reproductive tract variable for the oocyte source, sperm source and/or gestational carrier; a diagnostic test variable selected from the group consisting of endocrine diagnostic tests, liver function, renal function, electrolyte, cholesterol panel, imaging, metabolic panel, autoimmune disease panel, ovarian function, coagulation, recurrent pregnancy loss panel, prenatal panel, male endocrine panel, ovarian function or suppression diagnostic tests for gestational carrier or patient, and any diagnostic tests indicating the patient's organ function, any measure of nutrients or metabolites such as folate, and any folate metabolites; a clinical diagnosis variable related to a reproductive or sexual function selected from the group consisting of: tubal disease, recurrent pregnancy loss, endometriosis, uterine fibroid, congenital abnormalities affecting reproductive function, polycystic ovaries, polycystic ovarian syndrome, anovulation, irregular menstrual cycles, menorrhagia, ovarian cyst, ovarian masses, cervical abnormalities, diseases of the hypothalamus or pituitary glands or other hormonal dysfunction affecting reproductive functions, male factor, abnormalities in semen analysis, obstructive azoospermia, non-obstructive azoospermia, vas deferens abnormalities, erectile dysfunction, ejaculatory dysfunction, any gene variants associated with or causes or confers risk for reproductive dysfunction, clinical infertility, IVF failure, occurrence or recurrence of at least one IVF phenotype, pregnancy loss, recurrent pregnancy loss, obstetrical complications, fetal developmental defects, congenital birth defects, newborn illnesses; a clinical diagnosis variable related to a medical condition not related to a reproductive or sexual function; a microbiology variable affecting the oocyte source, sperm source and/or gestational carrier, selected from the group consisting of a variable relating to an infection with a known or unknown pathogen (e.g., Chlamydia trachomatis, Neisseria gonorrhorea, Trichomonas vaginalis, Syphilis, Bacterial vaginosis, Gardnerella vaginalis, Mycoplasma hominis, Ureaplasma urealyticum, anaerobes, Candidiasis, HIV, HPV, TB, Chancroid, Lymphogranuloma Venereum, herpes simplex virus, hepatitis B, and hepatitis C), an abnormal microbiome, an imbalance of normal flora, infection or relative levels of lactobacillus, gardnerella, Atopobium vaginae, megasphera, candida species or any micro-organisms, and a mix of micro-organisms causing or supporting abnormal pH levels in the reproductive tract; a sperm abnormality variable selected from the group consisting of an abnormal sperm count, abnormal count of motile sperm, abnormal percentage of sperm having certain motility measures, abnormal percentage or count of sperm having abnormal morphology, abnormal percentage or count of sperm having abnormal function, abnormal percentage or count of sperm having an abnormal amount of DNA, organelles, subcellular structures or any other detectable abnormal features; and a gene variant variable related to a clinical variable, demographic variable, reproductive history variable, reproductive tract variable, microbiology variable, microbiome variable, laboratory test variable, or clinical diagnosis variable for the oocyte source, the sperm source and/or the gestational carrier, or a sperm abnormality variable.

In some embodiments of any of the methods provided, the first model output is for the target patient's current or future IVF treatment.

In some embodiments, any of the methods further comprise applying, to the trained first model, at least one IVF treatment outcomes variable from the target patient for a current IVF treatment.

In some embodiments, any of the methods further comprise applying, to the trained first model, at least one IVF treatment outcomes variable from the target patient for one or more past IVF treatments.

In some embodiments, the first model output further provides one or more selected from the group consisting of a level of correlation or predictive relationship between two IVF phenotypes assigned to the target patient, and a level of correlation or predictive relationship between the target patient's concordance model output and the target patient's probability of an IVF failure.

In some embodiments, the at least one IVF phenotype assignment is a categorical variable selected from the group consisting of true or false; semi-quantitative categories indicating disease severity; and non-quantitative categories.

In some embodiments, the at least one IVF phenotype assignment is a numerical variable and wherein the numerical variable is a continuous variable quantifying from least to most severe.

In some embodiments, the at least one IVF phenotype assignment is a numerical variable and wherein the numerical variable is a discrete variable.

In some embodiments, the at least one concordance model output assignment for each for the unique patient-treatment incidences utilizes set thresholds in a quantitative scale or prognostic score thresholds based on a statistical comparison or a composite prognostic score generated by clinical outcomes prediction models.

In some embodiments, a plurality of performance metrics of the trained concordance model are compared to those of a control model (i) having no concordance model inputs and relying on mathematical averages for performance metrics, if the concordance model input uses one intermediate IVF outcome or clinical variable; (ii) having only one variable as a concordance model input, if the concordance model input uses two intermediate IVF outcome or clinical variables; or (iii) having fewer variables than the concordance model input, if the concordance model input uses more than two intermediate IVF outcome or clinical variables. In some embodiments, at least one of the plurality of performance metrics of the trained first model is compared to a corresponding performance metric of a control first model, wherein the control first model utilizes no input variables and outputs a mathematical average; and wherein the control first model comprises only one or fewer input variables than the trained first model.

In some embodiments, any of the methods further comprise evaluating a plurality of performance metrics of at least the trained first model or the trained concordance model using any method selected from the group consisting of AUC of an ROC curve, posterior log-likelihood, any log-likelihood, dynamic range, reclassification, calibration, precision, recall, and F1 score. In some embodiments, the evaluating the plurality of performance metrics utilizes an independent dataset that is not used in training.

In some embodiments, at least one of the plurality of performance metrics of the trained first model is compared to a corresponding performance metric of a control first model, wherein the control first model utilizes no input variables and outputs a mathematical average; and wherein the control first model comprises only one or fewer input variables than the trained first model.

In some embodiments, any of the methods further comprise executing any of the computer-executed methods and performing an assay method for detecting at least one gene variant variable correlating with one or more selected from the group consisting of the IVF outcome, the intermediate IVF outcome, and the IVF phenotype in the target patient.

In some embodiments, any of the methods further comprise executing any of the computer-executed methods and analyzing the at least one gene variant variable of the target patient and utilizing the gene variant variable in the inputting step (a).

In some embodiments of any of the methods provided, the training comprises utilizing a method selected from the group consisting of logic operations, mathematical operations, techniques in statistics, classification, regression, artificial intelligence, machine learning, generative artificial intelligence learning, natural language processing, unsupervised learning, supervised learning and any methods for building a prediction model, an algorithm, or an explainer.

In some embodiments of any of the methods provided, the at least one gene variant variable is selected from the group consisting of a single nucleotide polymorphism (SNP), copy number variation (CNV), DNA insertion, DNA deletion, DNA duplication, DNA inversion, structural chromosomal abnormality, DNA methylation, nuclear DNA variant, mitochondrial DNA variant, familial gene variant, de novo gene variant, polygenic gene variant, haplotype, variant of a chromosomal region, and any combination thereof. In various embodiments of any of the methods provided, the at least one gene variant variable is selected from the group consisting of a plurality of SNPs, CNVs, DNA insertions, DNA deletions, DNA duplications, DNA inversions, structural chromosomal abnormalities, DNA methylations, nuclear DNA variants, mitochondrial DNA variants, familial gene variants, de novo gene variants, polygenic gene variants, haplotypes, variants of a chromosomal region, and any combination thereof.

In another aspect, provided herein is a computer-implemented method for identifying and using diagnostic gene variants associated with or predictive for IVF outcomes. In some embodiments, the method comprises: (a) inputting, into a gene variant model, an IVF dataset from at least one group of unique patient-treatment incidences at one or more treatment centers, wherein the dataset comprises (i) data for generating at least one IVF phenotype assignment and at least one gene variant assignment, wherein the at least one IVF phenotype assignment is a discrete variable or a continuous variable, and wherein the at least one gene variant assignment is a categorical variable or a numerical variable; (ii) IVF outcomes data; (iii) intermediate IVF treatment outcomes data; (iv) clinical data; and (v) gene variant data; wherein the IVF outcomes data comprises at least one variable of an IVF outcome, wherein the IVF outcomes variable is an IVF outcomes failure variable, the IVF treatment outcomes variable is an IVF outcomes success variable, or the IVF outcomes variable is an IVF outcomes pending variable; wherein the intermediate IVF treatment outcomes data comprise a plurality of variables of one or more intermediate IVF treatment outcomes selected from the group consisting of patient oocyte status variables, patient embryo status variables, carrier status variables, and any combination thereof; wherein the clinical data comprises at least one clinical variable; wherein the gene variant data comprises at least one gene variant variable of an oocyte source, sperm source, and/or a gestational carrier; and (b) training the gene variant model using the at least one IVF phenotype assignment for each of the unique patient-treatment incidences and the at least one gene variant variable, thereby generating gene variant model outputs for each of the unique patient-treatment incidences, wherein each gene variant model output comprises one or more selected from the group consisting of (i) a probability of IVF failure and (ii) a probability of at least one occurring or recurring IVF phenotype; (c) applying, to the trained gene variant model, a target patient's data comprising: at least one intermediate IVF treatment variable, at least one clinical variable, at least one IVF phenotype assigned to the target patient, and at least one gene variant variable for the target patient; and (d) generating a trained gene variant model output for the target patient selected from the group consisting of the target patient's probability of IVF failure, the target patient's probability of at least one IVF phenotype occurring or recurring, and a combination thereof.

In some embodiments of any of the methods provided, the at least one gene variant variable is selected from the group consisting of a single nucleotide polymorphism (SNP), copy number variation (CNV), DNA insertion, DNA deletion, DNA duplication, DNA inversion, structural chromosomal abnormality, DNA methylation, nuclear DNA variant, and mitochondrial DNA variant, familial gene variant, de novo gene variant, polygenic gene variant, haplotype, and variant of a chromosomal region.

In some embodiments of any of the methods provided, the structural chromosomal abnormality is selected from the group consisting of chromosomal insertion, chromosomal deletion, chromosomal duplication, chromosomal inversion, balanced translocation, and Robertsonian translocation.

In some embodiments of any of the methods provided, the gene variant or the at least one gene variant is classified based on functional effect selected from the group consisting of normal function, likely normal function, functional variance of unknown significance, hypothetical function effect, likely functional effect, hypomorphic allele, functional effect, loss of function, and gain of function. In some embodiments of any of the methods provided, the gene variant or the at least one gene variant is classified based on a clinical importance selected from the group consisting of clinical variant of unknown significance, right match for phenotype, known risk factor, possible risk factor, variant-of-interest, pathogenic variant, penetrance-graded when known, pathogenic with high penetrance, and pathogenic with moderate penetrance. Detailed descriptions of variant classifications can be found in, for example, www.nature.com/articles/s41431-021-00903-z, the disclosure is herein incorporated by reference.

In some embodiments of any of the methods provided, the gene variant or the at least one gene variant is associated with, causes or confers risk for abnormal quality or function of uterine implantation, ovary, oocyte, embryo, sperm, fetal development and function, neonatal health and function, congenital anomalies, newborn illnesses, or adult-onset disease. In some embodiments of any of the methods provided, the gene variant or the at least one gene variant is identified by a method selected from the group consisting of GWAS, family pedigree, animal models, in vitro cell or tissue studies, studies utilizing genetic editing technology or related technology, case-control studies, whole genome sequencing, exome sequencing, SNP microarray, PCR, CGH array, utilizing gene variant databases and/or genetic ancestry algorithms or results, and any combination thereof. In some embodiments of any of the methods provided, the gene variant or the at least one gene variant is a potential pathogenic gene variant indicating a treatment option and/or a reproductive outcome prognosis, wherein the treatment options is selected from the group consisting of IVF, ICSI, in vitro maturation of oocytes, in vitro gametogenesis, use of alternative sperm source, oocyte source, embryo source or uterine source, use of a genetic editing technology, use of genetic analysis of oocytes, embryos or other cell types, and any combination thereof.

In another aspect, provided herein is a diagnostic test for a target patient comprising executing any one of the computer-implemented methods described herein for the target patient.

In another aspect, provided herein is a method of providing a clinician a probability of an IVF outcome occurring or recurring of a current or prospective IVF treatment for a target patient comprising: any one of the computer-implemented methods described herein for the target patient; and displaying on a device a user interface showing the probability of the IVF outcome for the target patient.

### DETAILED DESCRIPTION

Provided herein are computer-implemented methods that provide an objective determination of the probability of the occurrence or recurrence of one or more intermediate IVF outcomes. The methods also provide a prediction of the probability of a patient experiencing an IVF failure, such as no live birth after initiation of an IVF treatment. Additionally, the method provides a determination of the impact of an intermediate IVF outcome or an IVF phenotype or their recurrence on the probability of IVF failure.

In some embodiments, the computer-implemented method includes inputting an IVF dataset from IVF patients and treatments into a phenotype model (also referred to as a model or a first model or a predictive model) where the dataset includes IVF data, clinical data, and data for generating an IVF phenotype assignment (or IVF phenotype assignment data); training the model using IVF data and IVF phenotype assignment data to generate a trained phenotype model. The method also can include applying to the trained phenotype model target patient data which can include IVF data such as intermediate IVF treatment outcome data, clinical data, and IVF phenotype assignment data; and outputting a trained phenotype model output for the target patient such that the output includes a probability of having a failed IVF treatment, a probability of having an IVF phenotype, a probability of having a recurring IVF phenotype, or a combination thereof.

In other embodiments, the computer-implemented method includes inputting an IVF dataset from IVF patients and treatments into a phenotype model where the dataset includes IVF data, clinical data, and data for generating a concordance model output; generating a concordance model output for the IVF patients and treatments; and training the phenotype model using concordance model outputs for the IVF patients and treatments to generate trained phenotype model outputs. The method step of generating a concordance model output includes inputting into a concordance model a first set of concordance inputs including intermediate IVF treatment outcome or clinical data (e.g., variables) for the IVF patients and treatments and a second set of concordance inputs including intermediate IVF treatment outcome or clinical data for the IVF patients and treatments, training the concordance model, and outputting from the trained concordance model a concordance model output for the IVF patients and treatments.

The method can also include applying to the trained concordance model target patient data including a first concordance input and a second concordance input which are different, generating a concordance model output for the target patient, applying to the trained phenotype model the concordance model output, and generating a phenotype model output for the target patient such that the output includes a probability of having a failed IVF treatment, a probability of having an IVF phenotype, a probability of having a recurring IVF phenotype, a probability of having a concordance model output, a probability of having a recurring concordance model output, or a combination thereof.

In some embodiments, data for generating an IVF phenotype assignment and data for generating a concordance model output are the same or overlapping. In some embodiments, data for generating an IVF phenotype assignment and data for generating a concordance model output are different.

Provided herein is a computer-implemented method includes for identifying and using diagnostic gene variants associated with or predictive for IVF outcomes. The method includes inputting an IVF dataset from IVF patients and treatments into a gene variant model where the dataset includes IVF data, clinical data, gene variant data, data for generating at least one IVF phenotype assignment and at least one gene variant assignment, training the gene variant model using IVF phenotype assignment data and gene variant data to generate a trained gene variant model, applying to the trained gene variant model target patient data which can include IVF data such as intermediate IVF treatment outcome data, clinical data, gene variant data, and IVF phenotype assignment data, and outputting a trained gene variant model output for the target patient such that the output includes a probability of having a failed IVF treatment, a probability of having an IVF phenotype, a probability of having a recurring IVF phenotype, or a combination thereof.

In some embodiments, data is input into any of the methods including data for determining IVF outcomes based on current IVF treatment results, past IVF treatment results, or both; data for determining an intermediate IVF treatment outcome including oocyte, sperm and blastocyst (including embryo) data and patient data of the oocyte source, sperm source, and/or gestational carrier; data relating to clinical variables of the oocyte source, sperm source, and/or gestational carrier and data relating to gene variants variables of the oocyte source, sperm source, and/or gestational carrier. In some embodiments, the input data is a concordance model output. In some embodiments, the input data is a gene variant model output. In some embodiments, the input data is output data from a trained model.

The data described herein including IVF, clinical and gene variant data (variables) can be used in a one or more models (a phenotype model, a concordance model and a gene variant model) to determine the probability of a patient having a failed IVF treatment, the probability of a patient having an IVF phenotype, the probability of a patient having a concordance model output, or a combination thereof. In some embodiments, the patient's IVF phenotype is a recurring IVF phenotype such as a phenotype the patient has experienced in a previous IVF treatment. In some embodiments, the patient's IVF phenotype is an occurring IVF phenotype such as a phenotype the patient will experience in a current or future IVF treatment.

The method described herein is useful when it is performed after a patient has experienced a failed IVF treatment, and determining the probability of IVF failure in a next IVF treatment and the probability of experiencing an IVF failure phenotype (or more than one IVF failure phenotype) in the next IVF treatment is desired. In some embodiments, knowledge of the probability of having an IVF outcome can help to personalize treatment protocols, guide patient selection for treatment and further investigations related to IVF outcomes. The information obtained from the methods can be objectively compared across treatment centers, geographies, patient populations, and the like.

Set forth below is a description of what are currently believed to be preferred embodiments of the claimed invention. Any alternates or modifications in function, purpose, or structure are intended to be covered by the claims of this application. As used in this specification and the appended claims, the singular forms “a,” “an,” and “the” include plural referents unless the context clearly dictates otherwise. The terms “comprises” and/or “comprising,” as used in this specification and the appended claims, specify the presence of stated features, integers, steps, operations, elements, and/or components, but do not preclude the presence or addition of one or more other features, integers, steps, operations, elements, components, and/or groups thereof.

### Definitions

The term “provider” or “healthcare provider” can refer to a healthcare worker, healthcare professional such as a physician, fertility specialist, nurse, embryologist, technician, advanced practice practitioner, counselor, therapist, practitioner of alternative medical therapy, medical assistant, and any healthcare facility such as a hospital, clinic, or fertility center.

The term “IVF treatment” can refer to an IVF treatment cycle or assisted reproductive technologies with any of the steps including using medication to suppress ovarian follicular development and/or ovulation, controlled ovarian stimulation, egg retrieval, manipulation of eggs and sperm in vitro to obtain fertilized eggs or embryos, using intracytoplasmic sperm injection or in vitro fertilization to produce fertilized eggs or embryos, the in vitro handling of embryos such as culture, cryopreservation, thawing, and transfer of embryos to a woman's uterus, using medication to prepare and maintain a woman's uterine lining or endometrium for implantation and the maintenance of pregnancy, relying on a woman's natural cycle entirely or to various extents such as not using medication to suppress ovarian follicular development, ovulation or controlled ovarian stimulation, performing any processes on the sperm, oocyte, mature or fertilized eggs or embryos such as assisted hatching, calcium channel activation or deactivation or modulation, nuclear transfer, cytoplasmic transfer, mitochondrial transfer, collection of sperm or various stages of sperm maturation through physical intervention such as sperm aspiration, testicular biopsy, microsurgical epididymal sperm aspiration, testicular sperm aspiration, testicular sperm extraction, in vitro maturation of oocytes or sperm, in vitro gametogenesis, in vitro culture and maintenance of artificial womb, further manipulation of oocytes or embryos such as genetic testing, genetic manipulation such as gene or chromosome editing. An IVF treatment cycle can end at a live birth or no live birth.

The term “patient” or “subject” refer to a human oocyte source (e.g., provider or donor), human sperm source or donor, or a human gestational carrier. In some embodiments, the gestational carrier and the egg source are the same person. In many embodiments, the patient or target patient refers to a female human who is undergoing infertility or an in vitro fertilization cycle (an IVF treatment), has undergone infertility or IVF treatment, or is considering infertility or IVF treatment. In some embodiments, the patient is a woman and she and her male or female partner are the intended parents. In some embodiments, the patient is both the oocyte source and gestational carrier or just the oocyte source or just the gestational carrier or the patient is an intended parent who uses one or two other women as the oocyte source and gestational carrier. In some embodiments, the IVF treatment uses sperm from a male intended parent or a sperm donor. In some embodiments, the patient and her female partner are the intended parents undergoing reciprocal IVF in which the patient is the oocyte source and her female partner is the gestational carrier or the patient is the gestational carrier and her female partner is the oocyte source. In some embodiments, the patient is a woman and the intended single parent. In some embodiments, the intended parents are two male partners and one or both of them contribute sperm to an IVF treatment that uses oocytes from an egg donor and embryo(s) are transferred to the uterus of a gestational carrier. In some embodiments, the oocytes are procured as cryopreserved oocytes from an egg bank. In some embodiments, the embryos are donated by another patient or couple. In some embodiments, a component of the oocyte such as the mitochondria is contributed by another person and is inserted into the oocyte or embryo via in vitro manipulation.

The term “unique patient-treatment incidence” can refer to a female patient who has received no IVF treatment (e.g., never received IVF treatment), a female patient currently receiving an IVF treatment, a female patient who has completed an IVF treatment (e.g., completed an IVF cycle), a female patient who has cancellation of an IVF treatment (e.g., who started an IVF treatment and stopped or cancelled prior to the embryo transfer step), a current IVF treatment for a female patient, a previous IVF treatment for a female patient, a previous IVF treatment for the same female patient, and the like. In other words, data from a patient who has undergone 2 IVF treatments can be grouped such that data for the first treatment and data for the second treatment are assessed independently (e.g., separately or individually) or jointly (e.g., cumulatively).

The term “gestational carrier” refers to a patient herself or any woman intended to receive the embryo transfer and carry the gestation for the intended parents (who can be a heterosexual couple, same sex female couple, same sex male couple, or single person).

The term “IVF phenotype assignment” refers to a classification of one or more intermediate IVF treatment outcomes variables and/or one or more clinical variables. An IVF phenotype assignment can be any type of variable such as, but not limited to a discrete variable, continuous variable, categorical variable, numeric variable, quantitative variable, nominal variable, ordinal variable, binary variable, and the like.

The term “IVF phenotype” refers to a patient characteristic observed during an IVF treatment, such as, but not limited to, an intermediate IVF treatment outcome.

The term “gene variant assignment” refers to a classification of one or more gene variants (e.g., genetic variations) associated with or suspected of being associated with IVF failure, IVF phenotypes (e.g., IVF failure phenotypes), infertility, oocyte abnormalities, sperm abnormalities, reproductive abnormalities, gestational carrier abnormalities, oocyte source abnormalities, sperm source abnormalities, and the like. A gene variant assignment can be any type of variable such as, but not limited to a discrete variable, continuous variable, categorical variable, numeric variable, quantitative variable, nominal variable, ordinal variable, binary variable, and the like.

The term “concordance input” refers to data or variables that include (i) one or more (e.g., 1, 2, 3, 4, 5, 6 or more) intermediate IVF treatment outcomes or (ii) one or more (e.g., 1, 2, 3, 4, 5, 6 or more) clinical variables or (iii) at least one intermediate IVF treatment outcomes and at least one clinical variables. In other words, a concordance model input includes at least one intermediate IVF treatment outcomes or at least one clinical variables, such as, but not limited to, 1 intermediate IVF outcomes variable and 1 clinical variable; 2 intermediate IVF outcomes variables; or 2 clinical variables, and the like.

The term “concordance model” refers to a means to determine if a patient has a value of “expected” or “more than expected” or “fewer than expected” for a variable of an IVF treatment. For example, the variable can be for an oocyte source, sperm source, gestational carrier, treatment center, and the like. In some embodiments, a concordance model is applied to any intermediate IVF treatment outcomes described herein. In some instances, a concordance model is applied to an intermediate IVF treatment outcomes variable that is a continuous variable, a variable measured in integers, a categorical variable, an ordinal variable, an interval variable, or other type of variable.

The term “IVF outcome failure” refers to an IVF treatment that is initiated but does not result in a live birth due to any of: cancelled ovarian stimulation, cancelled oocyte retrieval, cancelled embryo transfer or no embryo available for transfer, or an at least one embryo transfer results in no live birth (such as but not limited to. due to no pregnancy, biochemical pregnancy loss, clinical pregnancy loss, or intrauterine fetal demise) and the like.

The term “pending IVF outcome” refers an outcome where IVF treatment has been initiated, remains in progress, and one or more of the following criteria has been met: (a) at least one condition selected from oocyte retrieval, in vitro fertilization or ICSI, embryo culture, embryo transfer, and/or embryo cryopreservation has not occurred, but is planned; (b) embryo transfer has occurred, but the serum B-HCG test result is not yet available; (c) at least one test selected from serum B-HCG test, ultrasound or other imaging test for gestational sac, fetal pole, fetal cardiac activity has not occurred, but is possible based on the steps that have occurred; (d) there is at least one cryopreserved embryo available for transfer; and (e) the IVF Treatment Outcome is not classified as Failure or Success.

The term “no live birth” includes, but is not limited to, one or more of the conditions such as, (a) serum B-HCG of the embryo transfer recipient is not detectable by 21 days after embryo transfer; (b) biochemical pregnancy loss in which the serum B-HCG of the embryo transfer recipient is initially detectable and positive and later, dropped to undetectable levels; (c) a gestational sac is visible on ultrasound and later disappears; (d) a fetal pole is detectable, fetal crown-rump length is measurable, fetal heart activity or any stage of fetal development is detectable on ultrasound and later, fetal development is arrested; (e) molar pregnancy is diagnosed; and (f) ectopic pregnancy is diagnosed.

The term “IVF outcome success” refers to a success after initiation of an IVF treatment such that the treatment resulted in a live birth (baby) after at least one transfer of at least one embryo into the patient or the intended gestational carrier.

The term “intermediate IVF treatment outcome” refers to one or more outcomes of an IVF treatment that has been initiated, excluding live birth, no live birth or pending. An intermediate IVF treatment outcome can be based on a quantifiable or a qualifiable value or score. An intermediate IVF treatment outcome can be cancellation of IVF treatment, the reason for IVF treatment cancellation, or details related to or causing IVF treatment cancellation.

The term “IVF failure” includes, but is not limited to, an IVF treatment resulting in no live birth, an IVF treatment resulting in no live birth and one or more intermediate IVF treatment outcomes described herein, an IVF treatment resulting one or more IVF outcomes described herein and one or more intermediate IVF treatment outcomes described herein, and any of the conditions described above in combination with at least one concordance model output. Without limitation, an IVF failure is an IVF outcome including no live birth, clinical pregnancy loss, biochemical pregnancy, implantation failure, no viable embryo, ectopic pregnancy, molar pregnancy and others.

### Method Inputs

The computer-implemented method provided herein utilizes a plurality of input data. In some embodiments, the input data (e.g., input variables) includes data regarding IVF outcomes, data regarding intermediate IVF treatment outcomes, clinical data from an oocyte or sperm source or a gestational carrier, gene variant data from an oocyte or sperm source or a gestational carrier or a related family member of the oocyte source, sperm source or gestational carrier, and data or model output generated from one or more models provided herein, such as a phenotype model, a concordance model, a gene variant model, or another IVF prognostic model.

Data regarding IVF outcomes includes, without limitation, an IVF treatment failure or no live birth with or without the specific circumstances (e.g. clinical pregnancy loss, biochemical pregnancy, implantation failure, no viable embryo, ectopic pregnancy, molar pregnancy), an IVF treatment success or a live birth, or a pending IVF treatment. Data of an IVF treatment failure can include data from one or more IVF treatment failures for one patient. Data of an IVF treatment success can include data from one or more IVF treatment successes for one patient. Data regarding an IVF treatment failure includes, without limitation, any of the IVF outcome failure variables described in detail below. Data regarding a pending IVF treatment includes, without limitation, any of the IVF outcome pending variables described in detail below. Data regarding an IVF treatment success includes, without limitation, the IVF outcome success variable described in detail below. Data regarding intermediate IVF treatment outcomes includes, without limitation, any of the intermediate IVF treatment outcomes variables described in detail below. Data regarding IVF phenotypes includes, without limitation, any of the intermediate IVF treatment outcomes variables described in detail below. Data regarding clinical data from an oocyte or sperm source or a gestational carrier includes, without limitation, any of the clinical variables described in detail below. Data regarding gene variant data from an oocyte or sperm source or a gestational carrier includes, without limitation, any of the gene variant variables described in detail below.

The computer-implemented methods described herein includes inputting an IVF data from IVF patients and treatments into a computer system to train and/or create any predictive model including a phenotype model, a concordance model, and a gene variant model.

### IVF Outcomes Variables

The IVF outcomes variables describes herein are part of an IVF dataset that is input into a model (e.g., a phenotype model, a concordance model, a gene variant model, and a first model) used to determine the probability of a patient having a failed IVF treatment, the probability of a patient having an IVF phenotype (including one or more than one phenotype), the probability of a patient having an concordance model output, or any combination thereof. Current or past IVF treatment results are used to determine IVF outcomes.

Provided herein is a variable corresponding to a successful IVF treatment that results in a live birth. Also provided herein are variables corresponding to an unsuccessful IVF treatment (failed IVF treatment) that fails to result in a live birth. In some embodiments, a variable corresponding to an IVF outcome failure includes a failure after initiation of an IVF treatment such that the treatment results in any one including cancelled ovarian stimulation, cancelled oocyte retrieval, no embryo transfer, no embryo cryopreservation, no live birth despite the transfer of at least one embryo, and the like. In some embodiments, a variable corresponding to an IVF outcome that is pending includes an outcome when an IVF treatment has been initiated, remains in progress, and any one of event occurs including, but not limited to, at least one condition selected from oocyte retrieval, in vitro fertilization or ICSI, embryo culture, embryo transfer, and/or embryo cryopreservation has not occurred, but is planned and deemed possible; embryo transfer has occurred, but the serum B-HCG test result is not yet available; at least one test selected from an serum B-HCG test, ultrasound or other imaging test for a gestational sac, fetal pole, fetal cardiac activity has not occurred, but is planned; at least one cryopreserved embryo is available for transfer; and an IVF outcome classified as either an IVF outcome failure or an IVF outcome success has not occurred.

In some embodiments, IVF outcome data is sourced from one or more treatment centers. In some embodiments, IVF outcome data for the group of unique patient-treatment incidences is from one treatment center. In some embodiments, IVF outcome data for the group of unique patient-treatment incidences is from 2 or more, e.g., 2, 3, 4, 5, or more treatment centers.

In some instances, an IVF outcome failure includes an IVF treatment that started and resulted in cancelled ovarian stimulation, cancelled oocyte retrieval, no embryo transfer, no embryo cryopreservation, or no live birth despite the transfer of at least one embryo, such that the “no live birth” outcome may be one of the following conditions: (a) serum B-HCG of the embryo transfer recipient was not detectable by 21 days after embryo transfer; (b) biochemical pregnancy loss in which the serum B-HCG of the embryo transfer recipient is initially detectable and positive and later, dropped to undetectable levels; (c) a gestational sac is visible on ultrasound and later disappears; (d) a fetal pole is detectable, fetal crown-rump length is measurable, fetal heart activity or any stage of fetal development is detectable on ultrasound and later, fetal development is arrested; (e) molar pregnancy is diagnosed; and (f) ectopic pregnancy is diagnosed.

In some instances, an IVF outcome success includes an IVF treatment that started and resulted in a live birth (baby) after at least one transfer of at least one embryo into the patient or the intended gestational carrier.

In some instances, an IVF pending outcome pending an IVF treatment that started and is in process, such that: (a) at least one of oocyte retrieval, in vitro fertilization or ICSI, embryo culture, embryo transfer, embryo cryopreservation has not occurred but is planned; (b) embryo transfer has occurred but the serum B-HCG test result is not yet available; (c) at least one of serum B-HCG test, ultrasound or other imaging test for gestational sac, fetal pole, fetal cardiac activity has not occurred but is planned; (d) there is at least one cryopreserved embryo available for transfer; and (e) the IVF outcome is not failure or success as defined above.

### Intermediate IVF Treatment Outcomes Variables

Oocyte, sperm and blastocyst (including embryo) data and patient data of the oocyte source, sperm source, and/or gestational carrier can be used to determine an intermediate IVF treatment outcome (also referred to as an intermediate IVF treatment outcomes variable) described herein. In other words, an intermediate IVF treatment outcome is an intermediate IVF treatment outcomes variable.

In some embodiments, an intermediate IVF treatment outcome includes a cancelled oocyte retrieval step due to presumed poor quality or ovarian follicular response as indicated by one or more of the following: (a) too few ovarian follicles of sufficient size (in some instances, as measured by imaging modality such as ultrasound); (b) inadequate ovarian follicular response such as in cases of suboptimal serum estradiol levels; and (c) the occurrence of premature ovulation (e.g. loss or absence of retrievable oocytes). In some embodiments, an intermediate IVF treatment outcome includes a cancelled oocyte retrieval step due to stopping ovarian stimulation to avoid worsening of ovarian hyperstimulation syndrome. In some embodiments, an intermediate IVF treatment outcome includes a cancelled oocyte retrieval step or stopping of ovarian stimulation due to incorrect administration of medication.

In some embodiments, an intermediate IVF treatment outcome variable includes an oocyte retrieval procedure resulting in (a) fewer than expected number of oocytes or no oocytes at all, (b) no oocytes despite the presence of growing or large ovarian follicles detected by imaging (such as ultrasound), (c) no oocytes due to a condition known as empty follicle syndrome; or (d) fewer than expected or no oocytes of sufficient quality.

In some embodiments, an intermediate IVF treatment outcome includes oocytes (whether different from expected in number and quality) failing to develop into 1-cell zygotes with 2 pronuclei (2PN) each. In some instances, failure to develop into such 1-cell zygotes results from: (a) oocytes having thick, thin, abnormal or absent zona pellucida; (b) abnormal morphology based on microscopically visibly or AI-imaging-detectable abnormalities or abnormal in the composition of proteins, protein modifications or ultrastructure; (c) failed or abnormal fertilization, which can result from oocyte or sperm abnormalities or dysfunction; and/or (d) failed intracytoplasmic sperm injection (ICSI).

In some embodiments, an IVF intermediate treatment outcome includes embryo arrest at one or more stages of development such that no or fewer than expected embryos reach the blastocyst stage. In some embodiments, an IVF intermediate treatment outcome includes failure of 2PN zygotes, if formed, to progress in their development from 1-cell to 2-cell, 4-cell, 8-cell, morula and blastocyst stages or any stages of in vitro maturation or in vitro gametogenesis. In some instances, embryos arrest at one or more stages of development such that no or few reach the blastocyst stage (which is the type of embryos considered to have sufficient potential for fetal development and are therefore the type of embryos most commonly transferred back to the uterus). In some embodiments, an IVF intermediate treatment outcome includes an outcome in which there are fewer than expected number of blastocysts or no blastocysts by day 5, day 6, or day 7 of culture. In some embodiments, an IVF intermediate treatment outcome includes an outcome in which if genetic screening such as, but not limited to, preimplantation genetic testing for aneuploidy (PGT-A) is performed, there are fewer than expected number of euploid blastocyst or no euploid blastocysts at all. In some embodiments, aneuploid (non-euploid) blastocysts have abnormal number of chromosomes in cells from the inner cell mass, trophectoderm or both. In some embodiments, aneuploid blastocysts have abnormalities in chromosome number that are complex; affect only one chromosome or more than one chromosome. In some embodiments, blastocysts are determined to be mosaic, meaning that some cells are euploid and some cells are aneuploid.

In some embodiments, an intermediate IVF treatment outcome includes an outcome in which 5%, 10%, 15%, 20%, 25%, 30% or more of the retrieved oocytes show abnormal morphology or morphology indicative of oocyte development arrest. In some instances, a retrieved oocyte with abnormal morphology has (a) an intact germinal vesicle, (b) is immature, (c) has an abnormal appearance such as fragmentation, vesicular appearance or granularity, (d) has a thin, thick, abnormal or absent zona pellucida, (e) cannot become a mature oocyte, or (f) cannot form normal zygote with two pronuclei (2PN).

In some embodiments, “abnormal morphology” is not specified and can include morphological features indicative of developmental arrest such as intact germinal vesicle, immature oocytes, fragmentation, vesicular appearance, granularity, zona pellucida that is thick, thin or absent, or any other abnormality.

In some embodiments, the abnormal morphology can be detected by humans (usually clinical embryologists), imaging analyzed by artificial intelligence, or oocytes deemed abnormal can be processed and analyzed by electron microscopy or other imaging techniques for abnormal morphology of subcellular structures.

In one embodiment, oocyte morphology includes any qualitative or quantitative measures of the size, volume, symmetry of the oocyte; relative distances or positions between any two or more organelles or subcellular structures; appearance or relative distances or positions between any two or more subcellular structures or organelles such as the number of mitochondria, the relative position of mitochondria to the nucleus, the position of the spindle or centrioles; the relative darkness/lightness of granules or certain organelles, etc. In one embodiment, oocytes deemed non-viable are subject to processing and visualization under the electron microscope or other imaging modality for qualitative or quantitative measures.

In some embodiments, an intermediate IVF outcome includes an outcome in which 5%, 10%, 15%, 20%, 25%, 30% or more embryos at each stage do not progress to the next stage. In some instances, such outcome includes 25% or more 2PNs embryos that do not develop to 2-cell embryos; 25% or more 2-cell embryos that do not develop to 4-cell embryos; 25% or more 4-cell embryos that do not develop to 8-cell embryos; 25% or more 8-cell embryos that do not develop to morulas; 25% or more morulas that do not develop to blastocysts; or 25% or more blastocytes that do not show expansion.

In some embodiments, an intermediate IVF treatment outcome includes an outcome in which 5%, 10%, 15%, 20%, 25%, 30% or more blastocysts are not Grade 5AA based on the Gardner score (Gardner et al., Fertil Steril. 2000; 73 (6): 1155-1158).

In some embodiments, an intermediate IVF treatment outcome includes when at each stage of oocyte or embryo development, 5%, 10%, 15%, 20%, 25%, 30% or more oocytes or embryos have an abnormal feature that can be: (a) detectable microscopically by a skilled healthcare provider, (b) detectable by AI-based imaging analysis, and (c) detectable by any imaging modality such as 2-dimensional or 3-dimensional modalities or biochemical modality. In some embodiments, the abnormal feature of the oocytes or embryos includes one or more abnormal ultra-structures, subcellular structures, or organelles, an abnormal cellular organization or pattern.

In some embodiments, an intermediate IVF treatment outcome includes when at each stage of oocyte or embryo development, 5%, 10%, 15%, 20%, 25%, 30% or more oocytes or embryos show accelerated or delayed timing in the division of one or more cells or in the progression from one developmental stage to the next, such as oocyte maturation-to-fertilization, ICSI-to-formation of 2PN, 2PN-to-4-cell stage, 4-cell-to-8-cell stage, 8-cell-to-morula stage, and morula-to-blastocyst stage.

In some embodiments, an intermediate IVF treatment outcome includes an outcome where 5%, 10%, 15%, 20%, 25%, 30% or more oocytes or embryos show an overall accelerated or delayed progression of two or more developmental stages. In some embodiments, an IVF intermediate treatment outcome includes an outcome where 5%, 10%, 15%, 20%, 25%, 30% or more oocytes or embryos are cultured in an in vitro culture media or environment with at least one abnormal metabolite or abnormal composition of metabolites.

In some embodiments, an intermediate IVF treatment outcome includes an outcome where 5%, 10%, 15%, 20%, 25%, 30% or more oocytes or embryos have (a) an abnormal, absent, insufficient, excessive biochemical function of any kind within the oocyte, sperm, embryo or oocyte-sperm interaction; (b) a presence, absence or an abnormal amount of at least one gene transcript, transcript variants, overall amount of gene transcripts, whether the transcripts are miRNA transcripts, mRNA transcripts, intracellular or secreted; or (c) a presence, absence or an abnormal amount of at least one protein or peptide, overall amount of proteins or peptides, protein modifications, whether the protein, peptide is intracellular, extracellular, secreted or contained in the phospholipid bilayer.

In some embodiments, an intermediate IVF treatment outcome includes an outcome where the thickness of the endometrial lining of the intended gestational carrier (e.g., embryo transfer recipient) is (a) less than expected or less than 7 mm at any two time points during medical or natural endometrial preparation within 14 days of the embryo transfer, or (b) less than expected or less than 7 mm at any time within 7 to 14 days of fresh or frozen-thawed embryo transfer; or considered to be inadequate to warrant cancellation of the current treatment, resulting in cancelled oocyte retrieval, cancelled embryo transfer or freezing of all embryos. In some embodiments, an intermediate IVF treatment outcome includes an outcome where the endometrium shows abnormalities on any imaging modalities including scarring, calcification, polyp(s), cyst(s), nodule(s), any hypodensities measuring over 1 mm, absence of trilaminar appearance, any fluid or mass in the endometrial cavity whether the treatment is cancelled or not. In some embodiments, an intermediate IVF treatment outcome includes none of the intermediate IVF outcomes described herein.

An intermediate IVF treatment outcome can be a feature, observation, or results at any step of the IVF cycle. In some embodiments, an intermediate IVF treatment outcome is a component, characteristic, aspect of an IVF phenotype.

In some embodiments, an intermediate IVF treatment outcome is related to a phenotypic feature of infertility. In one embodiment, phenotypic features of infertility as a disease and related and associated phenotypes such as pregnancy loss or developmental abnormalities observed or detected in utero that are described or if uncorrected, would lead to congenital anomalies, etc., such as those described in Online Mendelian Inheritance of Man (OMIM, Amberger 2019), Unified Medical Language System (UMLS, Bodenreider 2004), NCI Thesaurus (NCIt), Medical Dictionary for Regulatory Activities (MedRA, Brown et al. 1999), Medical Subject Headings (MeSH) and other medical dictionaries, ontologies and thesauruses, databases and publications.

### IVF Phenotypes

Provided herein are IVF phenotypes (also referred to IVF failure phenotypes) include any of the intermediate IVF treatment outcomes described herein. Additionally, any of the clinical variables described herein can contribute to an IVF phenotype. In some embodiments, any of the gene variant variables described herein can contribute to an IVF phenotype. In any of the IVF outcome failure variables described herein can contribute to an IVF phenotype.

Methods described herein are used to assign a patient to at least one IVF phenotype based on the phenotype model. At each and any step of an IVF treatment, the method provides a prediction of an IVF phenotype for a patient based on data from the patient.

In some embodiments, an IVF phenotype includes one or more variables selected from an IVF treatment outcome described herein and one or more variables selected from an IVF intermediate outcome described herein. In other embodiments, an IVF phenotype further includes one or more variables selected from the clinical variables described herein.

In some embodiments, a patient with one or more IVF treatments in the past may have the same or different IVF phenotype(s) associated with each of her past IVF treatments.

In some embodiments, the thresholds used to assign IVF phenotype is determined based on an analysis method selected from the group consisting of unsupervised learning, supervised learning, and other classification methods.

### Clinical Variables

In one embodiment, the clinical variable dataset includes family, medical and reproductive history. In one embodiment, the clinical variable dataset includes gene variants of immediate family members, extended family members, or both.

Clinical variables used in the method include, but are not limited to, baseline clinical or demographic variables such as body mass index (BMI) or any biometrics of the egg source or gestational carrier if not the female partner, female partner age, male partner age, gestational carrier (if not female partner) age, race, and ethnicity, reproductive history variables such as number of months or years of conception attempts; same-sex or heterosexual couple or single woman; history of pregnancy, live birth pregnancy loss; the number and type of past fertility treatments and outcomes, reproductive tract abnormalities for the female and/or male partners including abnormalities that are congenital, structural, developmental or acquired through other medical conditions such as infection, cancer, and iatrogenic causes, variables based on results from standard laboratory tests, variables based on clinical diagnoses related to reproductive or sexual function, variables based on clinical diagnoses related to other medical conditions, variables based on gene variant testing, variables based on microbiology or microbiome testing of at least one part of the female or male body, and variables based abnormalities in semen analysis parameters or known sperm dysfunction or conditions of the person contributing the sperm that may compromise IVF outcomes. In some embodiments, the clinical variable male factor includes severe sperm abnormalities in morphology, motility and count, azoospermia, the need for sperm extraction or testicular biopsy and donor sperm usage. In some embodiments, a clinical variable includes IVF treatment variables selected from the group consisting of: IVF treatment protocol, use of dietary supplements including vitamins, high dose folic acid, magnesium, selenium, calcium; ovarian suppression medication type, ovarian suppression medication dosage, ovarian suppression medication dose schedule, gonadotropin medication type, gonadotropin medication dosage, gonadotropin medication dose schedule, oral contraceptive pill type, oral contraceptive dosage, oral contraceptive dose schedule, gonadotropin releasing hormone agonist (GnRH agonist) type, GnRH agonist dosage, GnRH agonist dose schedule, GnRH antagonist type, GnRH antagonist dosage, GnRH antagonist dose schedule, luteinizing hormone (LH) type, LH dosage, LH dose schedule, human chorionic gonadotropin (HCG) type, HCG dosage, HCG dose schedule, progesterone or any progestin type, progesterone or progestin dosage, progesterone or progestin dose schedule, estradiol or any estrogen type, estradiol or estrogen dosage, estradiol or estrogen dose schedule, clomiphene, letrozole or any medication with similar action type, dosage of clomiphene, letrozole or any medication with similar action type, dose schedule clomiphene, letrozole or any medication with similar action type, any medication that controls, impacts, stimulates or suppresses ovarian function, ovarian follicle function, oocyte quality, endometrial receptivity or function, dose thereof, and dose schedule thereof. In some embodiments, a microbiology variable affecting the egg source, sperm source and/or gestational carrier can be a variable relating to an infectious agent that is related or unrelated to a sexually transmitted disease, microorganism(s) that are or are not part of the expected or normal flora, microorganisms that are or are not previously known to be associated with infertility or IVF treatment outcome.

In some embodiments, race and/or ethnicity are based on self-report by patient, patient's partner or both. In some embodiments, the self-reporting of race and/or ethnicity is analyzed from a detailed questionnaire soliciting self-reporting of the race and/or ethnicity of biological family members including one or more of biological parents, biological grandparents and biological great-grandparents. In some embodiments, race and/or ethnicity is further complemented or replaced by details such as place of origin, birth, childhood, youth and adulthood of self and/or one or more of biological parents, grandparents and great-grandparents, any immigration that occurred. In some embodiments, race and/or ethnicity is complemented or replaced by details such as the culture in the environment of the patient, partner, and/or one or more of biological parents, grandparents and great-grandparents. In some embodiments, place is country, region of a country, continent, or world region.

### Gene Variant Variables

Provided herein are computer-implemented methods for identifying and using diagnostic gene variants associated with or predictive for IVF outcomes. Such can be useful for determining associations between gene variants (genetic variants) and IVF phenotypes, intermediate IVF outcomes, and IVF outcomes predicted using the methods describe herein. In some embodiments, it is determined that one or more gene variants are associated with a female-focused, male-focused, oocyte-focused, sperm-focused, or embryo-focused IVF phenotype. In some embodiments, it is determined that one or more gene variants are associated with IVF failure or modify the risk of IVF failure. In some embodiments, it is determined that one or more gene variants are associated with one or more intermediate IVF treatment outcomes or IVF phenotypes described herein. In some embodiments, the gene variants are associated with an intermediate IVF treatment outcome or IVF phenotype before the patient has initiated on an IVF treatment or at any step of an IVF treatment.

In some embodiments of the computer-implemented methods for determining the probability of IVF failures and/or intermediate IVF outcomes, data for one or more gene variants are used to create and train one or more of the predictive models (e.g., phenotype model, concordance model, gene variant model, first model). In some embodiments, the computer-implemented methods for determining the probability of IVF failures and/or intermediate IVF outcomes are performed and an assay method is performed for detecting one or more gene variants correlating to the predicted IVF outcome, intermediate IVF treatment outcome, and/or IVF phenotype. In some embodiments, the computer-implemented methods for determining the probability of IVF failures and/or intermediate IVF outcomes are performed along with an analysis of one or more gene variants correlating to the predicted IVF outcome, intermediate IVF treatment outcome, and/or IVF phenotype.

The disclosure, in part, focuses on IVF phenotypes and associated gene variants as distinct from medical terms and clinical diagnoses that are typically described in the context of general infertility and reproductive phenotypes. Genetic variants have not been systematically investigated for their relevance in IVF outcomes or the occurrence or recurrence of IVF phenotypes. Genetic variants may be associated with reproductive, infertility, developmental defect, congenital anomaly phenotypes. Genetic variants include, but are not limited to, SNPs, CNVs; structural abnormalities of various sizes, from thousands of bases to major chromosomal rearrangements; insertions, deletions, balanced or Robertsonian translocations; DNA methylation, and mitochondrial DNA variants; and pathogenic or non-pathogenic genetic variants of implicated reproductive function genes, haplotypes or chromosomal regions. Detailed descriptions of exemplary types of genetic variants associated with other diseases such as cancer and developmental conditions or their prognoses can be found in, for example, (Zhang et al., Cancer Cell, 2020, 37(5), 639-654; Yuan et al., Cancer Cell, 2018, 34(4), 549-560; Momozawa et al, J. Natl Cancer Inst, 2020, 112(4), 369-376; Clause et al., Cell Genom, 2023, 3(2), 100258; Saia et al., Genes (Basel), 2023, 14(2), 500; Brownstein et al., Adv Genet (Hoboken), 2022, 4(1), 2200012; Escaramis et al., Briefings in Functional Genomics, 2015, 14(5), 305-314; Alkan et al., Nat Rev Genet, 2011, 12(5), 363-376; Chiang et al., Nat Genet, 2017, 49(5), 692-699; Houge et al., European Journal of Human Genetics, 2022, 30, 150-159; Bobyn et al., BMC Med Genet, 2020, 21, 92; Oak et al., Genome Med, 2020, 12(1): 51; Krainc and Fuentes, PNAS, 2022, 119(12), e2203033119; Huang et al., BMC Genomics, 2015, 16, 1093).

In one embodiment, the gene variants are limited to one or some specialized cell types. For instance, the specialized cells include oocytes, sperm, trophoblasts, uterine cells, immune cells and any somatic cells at any developmental stage, any stages of in vitro or in vivo maturation or differentiation. In one embodiment, the gene variants are present in the germline or in most cell types.

In one embodiment, the unbiased, systematic GWAS approach utilizing whole genome sequencing (WGS) or exome sequencing is taken to identify gene variants associated or predictive of IVF failure or IVF phenotype occurrence/recurrence, wherein the control may be drawn from general population controls from other studies or patients who are discharged from labor and delivery after childbirth or patients who had successful IVF treatments. In one embodiment, the methods used to identify gene variants prognostic for IVF outcomes are SNP microarrays, CGH arrays, PCR, WGS, exome sequencing. In one embodiment, the presence and quantity of RNA transcripts are measured to determine gene expression that is predictive or indicative or IVF outcomes or IVF phenotypes. In one embodiment, the genetic variants being investigated include variations in SNPs, CNVs, structural chromosomal abnormalities including insertions, deletions, inversions, balanced or Robertsonian translocations, DNA methylation, and mitochondrial DNA variants.

In some embodiments, gene variants are associated with one or more phenotypic features of infertility. In one embodiment, phenotypic features of infertility as a disease and related and associated phenotypes such as pregnancy loss, developmental abnormalities, congenital anomalies, etc., are sourced from Online Mendelian Inheritance of Man (OMIM, Amberger 2019), Unified Medical Language System (UMLS, Bodenreider 2004), NCI Thesaurus (NCIt), Medical Dictionary for Regulatory Activities (MedRA, Brown et al. 1999), Medical Subject Headings (MeSH) and other medical dictionaries, ontologies and thesauruses, databases and publications. In one embodiment, natural language processing (NLP) or other methods are used to extract or transform published data on genetic variants into structured data or database (e.g. Allot et al. 2018, Lee 2021).

In one embodiment, identification of gene variants correlating or predicting IVF outcomes or the occurrence/recurrence of certain IVF phenotypes alone or together with other clinical variables or intermediate IVF outcomes variables, explain partially or wholly, the mechanism of IVF failures previously thought to be or meeting the criteria of “recurrent implantation failure” or unexplained infertility.

In one embodiment, inclusion of gene variants for analysis and training in the phenotype model is informed by pre-existing databases such as National Center for Biotechnology Information (NCBI, Sayers et al. 2022), GeneCards (Stelzer et al. 2016), Hugo Gene Nomenclature Committee (HGNC, Bruford et al. 2007), Kyoto Encyclopedia of Genes and Genomes (KEGG, Kanehisa et 1. 2016), UniProt (UniProt 2023), dbSNP, and Gene Ontology (GO, The Gene Ontology 2019). In one embodiment, such inclusion of gene variants is informed by genomics data or phenotype-genotype correlations from human, mouse or other organisms.

In some embodiments, gene variants correlated with or prognostic for IVF outcomes or IVF phenotype or concordance model are determined by microarray (Balague-Dobon et al. 2022). In some embodiments, mutations or gene variants related to IVF phenotype or IVF outcomes are co-inherited in groups (e.g., Jiang et al. 2022). In one embodiment, the present invention is used for generating an IVF phenotype, genotype and model database comprising a list of IVF failure phenotype-associated genetic variants for validating against DNA from patients who had IVF failure phenotypes as determined by the methods describe herein. The database including list of IVF failure phenotype-associated genetic variants can be used in the methods for predicting IVF outcomes. In some embodiments, the method described herein such as the gene variant model and the phenotype model are useful for identifying IVF failure phenotype-associated genetic variants.

In some embodiments, one or more aspects of the computer-implemented method such as the first model, intermediate IVF outcomes dataset, IVF phenotypes, clinical variable dataset, concordance model, gene variant model and IVF outcomes dataset are analyzed with genetic ancestry data, genetic ancestry algorithm(s), genetic variants specific to certain ancestral groups to ascertain any potential increased or decreased frequency of certain clinical variables or intermediate IVF outcomes variables or any IVF phenotypes or other gene variants associated with one or more ancestral groups. In some embodiments, one gene variant variable is ancestry group based on the output generated by a genetic ancestry algorithm. In some embodiments, the gene variant model uses at least two gene variant variables, one of which is the genetic ancestry group such that testing of association, correlation or prognostic impact of a gene variant includes adjustment for genetic ancestry.

In some embodiments, analysis of one or more aspects of the computer-implemented method such as the first model, intermediate IVF outcomes dataset IVF phenotypes, clinical variable dataset, concordance model, gene variant model and IVF outcomes dataset establishes certain IVF phenotypes with or without associated gene variants as rare diseases. In some embodiments, intermediate IVF outcomes that may appear to one skilled in the art to be common are found to be rare diseases sharing similar clinical variables or intermediate IVF outcome variables or IVF phenotypes while varying in associated gene variants indicative of variation in the underlying disease mechanisms.

In some embodiments of the methods described, gene variants include SNPs such as OZEMA 14 (a mutation called Oocyte/Zygote/Embryo Maturation Defect 14 in the human CDC20 gene) located in chromosome 1p34. Case studies and case series in the literature reported unrelated patient-treatment incidences as well as genetically related patients with the OZEMA 14 mutation, in addition to intermediate IVF outcomes such as having all or most oocytes arrested at germinal vesicle or metaphase I (MI) stage, early embryo developmental arrest, fertilization failure, and unexplained recurrent pregnancy loss (Zhao et al., Protein Cell, 2020, 11:921-927). The reported variants were not found in most public variant databases or present at low minor allele frequency. The reported variants are examples of SNPs causing missense mutations (e.g. one amino acid change in the resulting protein) in the CDC20 gene including homozygous Y228C substitution, L439R substitution, R322Q substitution, heterozygosity for a missense mutation causing A211T substitution, and truncating mutations in CDC20. In some embodiments, the gene variant is determined to occur de novo as it is not found in other related family members. In some embodiments, the gene variant is found in other related family members in patterns consistent with autosomal recessive, autosomal dominant, X-linked recessive, X-linked dominant, or Y-specific patterns of inheritance. In some embodiments, the gene variant is present in a haplotype block in patients who are not genetically related family members and in that case and the frequency of the gene variant is higher in a specific ancestry group or population sharing the same founder. Some of the OZEMA 14 mutations were found to be inherited in a pattern consistent with autosomal recessive inheritance and one mutation was found to be present in a haplotype block consistent with founder mutation in the Chinese population (Xu, J Assist Reprod Genet, 2021, 38, 2219-2222). In some embodiments, gene variants associated with premature ovarian failure such as the CNVs reported by Tsuiko et al. (Tsuiko et al., Human Reproduction, 2016. 31 (8), 1913-1925) are analyzed by gene variant model training for predictive impact on IVF failure or association with intermediate IVF outcomes or IVF phenotype. Those CNV examples included 11 novel microdeletions, microduplications in genes FMN2 and SGOL2 essential for meiotic progression; TBP, SCARB1, BNC1, ARFGAP3 important for follicular growth and oocyte maturation; and hemizygous microdeletions of SYCE1 and CPEB1 with important meiotic functions. In some embodiments, gene variants include CNVs associated with or causative of male infertility and spermatogenic failure (Balkan J Med Genet 2019), Y chromosomal microdeletions associated with non-obstructive azoospermia previously identified by GWS and NGS (Liu et al, 2016. Sci Rep 2016) are analyzed and trained with IVF dataset, intermediate IVF phenotype and IVF outcomes. In some embodiments, CNVs in the form of microdeletions of the X chromosome associated with increased risk of premature ovarian failure, identified using array CGH and NGS techniques, are used in gene variant model training for association with or prediction for IVF phenotype(s), IVF treatment outcome failure, intermediate IVF outcomes or concordant model. Examples of deletions (0.16 to 3.72 MB) affecting genes TSPAN12, CLN8, ARHGEF10, ND1, MYH11, ABCC6 on autosomal and X chromosomes were associated with female infertility; duplications (1.32 to 2.4 MB) affecting various genes in autosomal and X chromosomes were associated with female infertility. In some embodiments, gene variants are chromosomal translocations that have been reported to be associated with IVF failure and recurrent pregnancy loss. For example, using cytogenetic studies, translocations including reciprocal and Robertsonian translocations were found in 1.4% individuals and 3.2% of couples with at least 10 embryos, no other clinical abnormalities and failed IVF after embryo transfer, whereas they were found in 4.1% of individuals and 9.2% couples with recurrent miscarriage in a study of 514 patients with IVF failure and 319 patients with recurrent pregnancy loss (Stern et al, Hum Repro 1999; Zeng et al, Sci Reports, 2023). Those chromosomal translocations were not tested for their association or prevalence among a broader group of patients.

In some embodiments, the methods described herein are useful for the discovery of novel gene variants associated with one or more of IVF failure, IVF phenotypes, and intermediate IVF treatment outcomes as set forth and defined herein. In some embodiments, the gene variants provided can be used in methods described herein for predicting the probability of one or more of IVF failure, IVF phenotypes, and intermediate IVF treatment outcomes as set forth and defined herein.

### Phenotype Model

A phenotype model can be created by inputting IVF data into a computer system, and the IVF data can include, but is not limited to, IVF phenotype assignment data, concordance model outputs, IVF outcomes data, intermediate IVF treatment outcomes data, clinical data, and optionally gene variant data from unique patient-treatment incidences sources from one or more treatment centers. Also, an untrained phenotype model can be trained using similar or different IVF data.

In the provided methods, a patient is assigned one or more IVF phenotypes described herein based on the phenotype model. As an exemplary embodiment, a patient can be assigned one or more specific IVF phenotypes if the patient had a failed IVF treatment with a biochemical pregnancy loss and an oocyte retrieval that resulted in a fewer than expected number of oocytes such that the specific IVF phenotypes include (1) an IVF outcome failure due to biochemical pregnancy loss, and (2) an intermediate IVF treatment outcome where the oocyte retrieval procedure resulted in a fewer than expected number of oocytes or more oocytes. In some embodiments, a patient experiences more than one intermediate IVF treatment outcomes and is assigned to more than one IVF phenotype.

In some instances, the phenotype model can assign at least one IVF phenotype to each unique patient or each unique patient-treatment incidence who has initiated at least one IVF treatment. In some instances, the phenotype model can assign TRUE vs. FALSE for at least one IVF phenotype by applying one or more sets of operations including, but not limited to, logic operations.

A patient's data can be inputted and processed by the phenotype model to obtain at least one IVF phenotype assignment, and the assignment is applied to the model described herein to generate a model output.

In some embodiments, the model undergoes training such as, but not limited to, simple mathematical operations including calculating the percentage of cases that would have a certain outcome. In other embodiments, the model training uses a method selected from the group consisting of logic operations, complex mathematical operations, and techniques in statistics, classification, regression, artificial intelligence, machine learning, generative artificial intelligence learning, natural language processing, unsupervised learning, and supervised learning.

### Concordance Model

A concordance model can be created by inputting concordance input data into a computer system, and each concordance input comprises at least one intermediate IVF treatment outcome or clinical variable from unique patient-treatment incidences sources from one or more treatment centers. Also, an untrained concordance model can be trained using similar or different concordance input data. Provided herein a concordance model that is created and applied to provide an objective determination if an intermediate IVF outcome should be expected, less than expected, or more than expected. In some embodiments, the concordance models are used to determine if a patient has favorable or unfavorable discordance. In many embodiments, the concordance models are used to determine if a patient has favorable or unfavorable concordance.

A concordance model can be applied to ascertain if a patient or unique patient-treatment incidence has “more than expected” or “fewer than expected” for one or more variables such as, but limited to the number of ovarian follicles, oocytes, 2PNs blastocytes, embryos of a certain developmental stage, etc. In some instances, the variables can be the number of oocytes retrieved, the number of mature oocytes, the number of oocytes with a certain quality, the number of blastocysts, the number of certain types of blastocysts, and the like.

A concordance model output can be generated for one or more intermediate IVF treatment outcomes described herein from patient data, treatment data, or a combination thereof. In some embodiments, a concordance model is used or applied for one intermediate IVF treatment outcome described herein. In some embodiments, a concordance model is used or applied for at least one intermediate IVF treatment outcome, e.g., 1, 2, 3, 4, 5, or more intermediate IVF treatment outcomes. In some embodiments, one can apply a concordance model for two or more intermediate IVF treatment outcomes. In some embodiments, a concordance model is used or applied for any intermediate IVF treatment outcome that is quantifiable.

In some embodiments, a concordance model used in the present method includes, but is not limited to, an oocyte yield prediction vs. actual oocyte yield concordance model, a blastocyst yield prediction vs. actual blastocyst yield concordance model, a blastocyst grade score prediction vs. actual blastocyst grade score concordance model, a 2PN zygote yield prediction vs. actual 2PN zygote yield concordance model, a 4-cell embryo yield prediction vs. actual 4-cell embryo yield concordance model, a 8-cell embryo yield prediction vs. actual 8-cell embryo yield concordance model, a morula yield prediction vs. actual morula yield concordance model, a fertilization prediction vs. actual fertilization concordance model, an ICSI prediction vs. actual ICSI concordance model, a genetic screening prediction vs. actual genetic screening concordance model, an oocyte morphology prediction vs. actual oocyte morphology concordance model, an oocyte morphology prediction vs. actual oocyte morphology concordance model, an oocyte morphology prediction vs. actual oocyte morphology concordance model, an embryo morphology prediction vs. actual embryo morphology concordance model, an embryo development prediction vs. actual embryo development concordance model, an embryo development timing prediction vs. actual embryo development timing concordance model, a development progression prediction vs. actual development progression concordance model, an in vitro culture condition prediction vs. actual in vitro culture condition concordance model, a sperm function prediction vs. actual sperm function concordance model, an oocyte genetic variant prediction vs. actual oocyte genetic variant concordance model, an oocyte protein prediction vs. actual oocyte protein concordance model, an oocyte biomarker prediction vs. actual oocyte biomarker concordance model, an endometrium prediction vs. actual endometrium concordance score an egg provider prediction vs. actual egg provider concordance score, a gestational carrier prediction vs. actual gestational carrier concordance score, a sperm provider prediction vs. actual sperm provider concordance score, and the like. In some embodiments, a concordance model is generated based on one or more IVF patient-specific factors, including but not limited to, genetic variants, microbiome biomarkers, infection biomarkers, environmental factors, and health factors. In some instances, IVF patient-specific factors are specific to oocyte providers, sperm providers, and gestational carriers. In some embodiments, a concordance model is generated for any intermediate IVF outcome or IVF failure phenotype described herein. In some instances, concordance models are generated from data one or more treatment centers. Concordance models can also be generated from data of a specific time period or multiple time periods.

In some instances, one or more concordance model outputs predict an IVF outcome failure. In some instances, one or more concordance model outputs are associated with an IVF outcome failure. A concordance model output can be directed to a recurring intermediate IVF outcomes or IVF failure phenotype in a patient. A concordance model output can be directed to a non-recurring intermediate IVF outcomes or IVF failure phenotype in a patient.

In an exemplary embodiment, an “oocyte yield prediction-oocyte yield concordance model” includes scores (values) corresponding to a Predicted Oocyte Yield, a Higher-than-Predicted Oocyte Yield, and a Lower-than-Predicted Oocyte Yield. An “oocyte yield prediction” concordance model output can be computed based on one or more algorithms using one or more predictors of oocyte number and/or quality described herein. For instance, a concordance model output for mature oocyte yield (more specifically, the number of mature oocytes) is generated by: (1) using a dataset containing a score for a clinical variable described herein and the number of mature oocytes retrieved (oocyte yield), (2) applying at least one statistical method to determine the relationship between the score of the clinical variable and the oocyte yield, and (3) determining whether the observed oocyte yield is less than expected, greater than expected, or as expected based on the score for the clinical variable.

In some embodiments, the dataset includes scores for 2 or more clinical variables described herein. In some embodiments, the dataset includes a score corresponding to 2 or more clinical variables. In some embodiments, the statistical methods that are useful include any statistical method recognized by one of ordinary skill in the art such as, but not limited to, logistic regression and linear regression, any machine learning method recognized by one of ordinary skill in the art such as, but not limited to, GBM, Xboost, and random forest, any statistical method which can be applied to a distribution including those recognized by one of ordinary skill in the art such as, but not limited to, binomial, poisson, chi square, F distribution, and the like. In some embodiments, the determining step is based on any way to parse, distribute, or stratify data. In some embodiments, the determination is based on X-tile (e.g. decile, quartile, tertile, other percentile, etc.) groupings of a clinical variable. In some embodiments, the determination is based on a statistical distribution such as 1 standard deviation, 2 standard deviations, 3standard deviations, etc. In some embodiments, the determination is based on a degree of confidence interval of prediction such as a 90%, 91%, 92%, 93%, 94%, 95% confidence interval, etc. In some embodiments, the determination is based on other distributions, intervals, and cut-offs recognized by one of ordinary skill in the art. In yet other embodiments, the determining step is based a composite prognostic score or composite prognostic tier such as those described in detail in, for example, U.S. Pat. Nos. 9,458,495 and 10,438,686, the contents of which are herein incorporated by reference in their entireties.

Solely as an example, if the oocyte yield for a patient group or a treatment group is in the lowest 10th-tile or decile for a given clinical variable score and the cut-off is set at below 25%-tile for a “less than expected” oocyte yield, then the number of mature oocytes retrieved from a subject patient that is below the 25%-tile will be determined to have an oocyte yield concordance model output of “less than expected”. In another example, if an oocyte yield of 3 or fewer is below the 30%-tile and the cut-off is set at 30%-tile, then an oocyte yield of 2 from a subject patient is determined to be “less than expected.”

In some embodiments, generating a concordance model includes validation of the scale using an independent dataset from, for example, the same or different patient population, the same or different treatment center(s), and/or the same or different geographical location of either the patient population(s) and/or the treatment center(s).

In some embodiments, generating a concordance model includes comparing model performance parameters of the scale against an AGE-only control model or an AVERAGE-only control model. In some instances, performance parameters are analyzed for a concordance model that uses N clinical variables and compared against a control model that uses M clinical variables provided that N is greater than M. In some embodiments, the performance parameters of several models are compared.

In some embodiments, generating a concordance model includes determining one or more model performance parameters related to the scale's applicability to predicting occurrence or recurrence of a specific intermediate IVF outcome or an IVF failure. In some instances, the model performance parameter includes AUC of the ROC curve, log-likelihood, posterior log-likelihood of odds ratio compared to AGE model (PLORA) or AVERAGE of the dataset, dynamic range, reclassification, precision, recall, and F1 score (a composite of precision and recall).

In one embodiment, the concordance model thresholds are established or trained using percentiles, deciles, tertiles, or quartiles; measures of statistical distribution such as 1 or 2 standard deviations; a certain level of confidence interval such as 95% confidence interval; unsupervised classification such as k-means; classification methods such as R-part, MART, CART; diagnostic criteria established by expert consensus; or a combination thereof.

In some embodiments, any prediction of concordance models can be based on one or more algorithms using predictors.

In some embodiments, concordance models are trained using one or more selected from the group consisting of simple mathematical operations, logic operations, complex mathematical operations, and techniques in statistics, classification, regression, artificial intelligence, machine learning, generative artificial intelligence learning, natural language processing, unsupervised learning, and supervised learning.

In one embodiment, IVF phenotypes and concordance model thresholds and their associated prediction or correlation of IVF treatment outcomes, occurrence or recurrence of IVF phenotypes, are recorded and tracked in a database, wherein usage of the database for research, investigations of disease mechanisms, development of devices and therapeutics, clinical service requires a license. In one embodiment, such an IVF phenotype and model database provides results that are generalizable to different locations and patient populations without local validation. In one embodiment, such an IVF phenotype and model database provides results that are specifically validated for certain locations and/or patient populations. In one embodiment, the above IVF phenotype and model database provides information or specificity based on the presence of certain gene variants. In one embodiment, the database is an IVF phenotype and genotype model database.

### Gene Variant Model

A gene variant model can be created by inputting IVF data into a computer system, and the IVF data can include, but is not limited to, IVF phenotype assignment data, gene variant assignment data, IVF outcomes data, intermediate IVF treatment outcomes data, clinical data, and optionally gene variant data from unique patient-treatment incidences sources from one or more treatment centers. Also, an untrained gene variant model can be trained using similar or different IVF data. In some embodiments, the gene variant model is trained using one or more selected from the group consisting of simple mathematical operations, logic operations, complex mathematical operations, and techniques in statistics, classification, regression, artificial intelligence, machine learning, generative artificial intelligence learning, natural language processing, unsupervised learning, and supervised learning.

In one embodiment, the patient using or being served by the gene variant model include the patient and the patient's family members. In some cases, use of immediate family members' and/or extended family members' gene variant status improves precision or the scope of the gene variant model output.

### Method Outputs

If a patient is currently in IVF treatment, the method can predict the probability of the patient having one or more intermediate IVF treatment outcomes and optionally, the probability of the patient having an IVF failure, and vice versa. If a patient has a past failed IVF treatment and is not currently in treatment, the method can predict the probability of the patient having a recurring IVF phenotype, a first occurrence of an IVF phenotype not previously experienced and optionally, the probability of the patient having an IVF failure. If a patient has a past successful IVF treatment and is not currently in treatment, the method can predict the probability of the patient having a recurring IVF phenotype, a first occurrence of an IVF phenotype not previously experienced and optionally, the probability of the patient having an IVF failure.

In some embodiments, a model output provides the cumulative probability of IVF failure, wherein cumulative probability refers to the probability of having no live birth from 1, 2, 3 or more consecutive IVF treatments or IVF treatment attempts (if one or more IVF treatments is cancelled or cannot be completed). In some embodiments, a model output provides the conditional probability of IVF failure, wherein the probability of have no live birth from IVF is based on knowing the patient has any one or more of certain clinical variables, past IVF treatment outcome, intermediate IVF outcome, IVF phenotype, concordance model, or gene variant a priori.

In some embodiments, the model outputs are presented in visual graphics such as a pie chart, bar graph or infographics-styled pictures. In some embodiments, one or more of the steps of entering the target patient's data and providing the model output for the target patient are performed in an interactive format which can be supported by videos, cartoons, audio, text, email, conversations with humans, and conversations with a machine.

In some embodiments, the input data from the target patient is extracted automatically from the electronic medical record (EMR) system, the model output for the target patient is automatically transmitted to the EMR system, the extraction and/or transmission of data or model output is semi-automatic, manual, delivered to other medical devices such as ultrasound machine. In some embodiments, the EMR is a decision support system used by providers, patients, consumers, other professionals or payers such as insurance companies, benefits companies, governments.

In one embodiment, the model output or the other outputs described herein including the phenotype model output, the concordance model output, and the gene variant model output informs the design of discovery including the types of human subjects to be recruited in a research study, the types of human subjects from whom to collect genomic DNA or other biological samples. In one embodiment, the model output or the other outputs described herein including the phenotype model output, the concordance model output, and the gene variant model output informs the pricing strategy, reimbursement strategy, warranty programs, risk-based pricing, value-based pricing, health insurance plan or health benefits plan designs, government support of IVF services.

In some embodiments, the model output explains occurrence or recurrence of IVF failure, clinical infertility, recurrent pregnancy loss, prior congenital anomalies, neonatal disease(s), or other clinical attributes of the patient, patient's partner or related family members. In some embodiments, the model output is a diagnostic test or part of a diagnostic test or evaluation for occurrence or recurrence of IVF failure, clinical infertility, recurrent pregnancy loss, prior congenital anomalies, neonatal disease(s), or other clinical attributes of the patient, patient's partner or related family members.

In some embodiments, the model output supports, informs or is a part of the provider-patient conversations and counseling; patient or consumer education, provider education. In some embodiments, one or more embodiments of the invention is used to inform selection of reproductive partner or the source of eggs and sperm and gestational carrier. In some embodiments, the method is used in a consumer product or consumer business model to inform selection of reproductive partner or the source of eggs and sperm and gestational carrier.

In some embodiments, the model output or analysis of intermediate IVF outcomes inform or support clinical operations including quality control, monitoring, evaluation of IVF outcomes, operational efficiency and cost-efficiency. In some embodiments, the model output alone or in combination with other information is used to determine whether clinical protocols and/or clinical embryology laboratory processes are performing as expected or if investigations or improvements are needed or clinical embryology lab protocols or processes warrant review and troubleshooting. In some embodiments, the model output is used to evaluate or provide benchmarks for the performance of certain products or equipment used by the embryology lab such as embryo culture media, incubator, any equipment that is used for the in vitro manipulation of oocytes and sperm, vitrification, cryopreservation or thaw. In some embodiments, the model output is used to evaluate or provide benchmarks for the performance of cryopreservation storage facilities, egg and sperm banks.

In some embodiments, the method is used in the design, execution and analysis of clinical trials. In some embodiments, the method is used to improve efficiency, cost-efficiency and likelihood of detecting efficacy of the intervention in a clinical trial.

In some embodiments, the method is used to support, expand, refresh, preserve, maintain or sustain a local or national population. In some embodiments, the population sustenance, maintenance, preservation or expansion supported by one or more aspects of the method is of economic utility to the population or government. In some embodiments, one or more aspects of the method is used to found or support the founding of a new population or inform the design and strategy of new population founding. In some embodiments, the use of the method is to support new population founding is part of a strategy to improve distribution of human populations, the environment, safety, health, economics of the populations or their support infrastructure. In some embodiments, the use of one or more aspects of the methods is to support space travel, navigation, migration or extra-terrestrial living.

In some embodiments, the frequency of certain model output and IVF phenotypes is evaluated and compared among different patient populations, locations, clinics. This frequency is used to inform or support the research design including recruitment priority and inclusion of certain clinics, patient populations, locations for research study. In some embodiments, one or more models is validated by testing the model on an independent dataset, an in-time dataset, an out-of-time dataset, cross validation, and a dataset of patients from a different location.

### Example 1. Using IVF Phenotype Assignments in a Phenotype Model

The method described herein include inputting into a model a series of datasets from at least one group of unique patient-treatment incidences, such as for Patient 1-Patient 6 and 1^{st} and 2^{nd} IVF treatments in Table 1. Patient 1 undergoes a 1^{st} IVF treatment which is completed and results in no live birth. Patient 1 undergoes a 2^{nd} IVF treatment which is completed and results in a live birth. Such data is included in the IVF treatment outcomes dataset. An intermediate IVF treatment outcome dataset can include data regarding a patient oocyte variable including if an oocyte retrieval is cancelled and if cancelled, the reason for the cancellation. A clinical variable dataset of the series can include patient age.

TABLE 1

| | Series of Datasets | | | | | |
|---|---|---|---|---|---|---|
| | | | | Intermediate IVF Treatment | | |
| | IVF Treatment | | | Outcome Dataset | | |
| Unique | Outcomes Dataset | | | | If oocyte | Clinical Variable |
| Patient- | | | IVF | Is oocyte | retrieval is | Dataset |
| Treatment | IVF | | Treatment | retrieval | cancelled, what | Patient Age |
| Incidences | Treatment | Completed | Outcome | cancelled? | is the reason | (using own eggs) |
| Patient 1 | 1 | Completed | No Live Birth | No | n/a | 33 |
| Patient 1 | 2 | Completed | Live Birth | No | n/a | 34 |
| Patient 2 | 1 | Completed | No Live Birth | Yes | OHSS | 38 |
| Patient 2 | 2 | Completed | Live Birth | No | n/a | 39 |
| Patient 3 | 1 | Completed | No Live Birth | Yes | too few ovarian | 32 |
| | | | | | follicles meet | |
| | | | | | size criteria | |
| Patient 3 | 2 | Completed | No Live Birth | No | n/a | 32 |
| Patient 4 | 1 | In Progress | Pending | No | n/a | 33 |
| Patient 5 | 1 | Completed | No Live Birth | Yes | too few ovarian | 33 |
| | | | | | follicles meet | |
| | | | | | size criteria | |
| Patient 5 | 2 | Completed | Live Birth | No | n/a | 33 |
| Patient 6 | 1 | Completed | No Live Birth | Yes | too few ovarian | 34 |
| | | | | | follicles meet | |
| | | | | | size criteria | |
| Patient 6 | 2 | Completed | No Live Birth | Yes | too few ovarian | 34 |
| | | | | | follicles meet | |
| | | | | | size criteria | |

The model can be trained using IVF phenotype assignments and IVF treatment outcomes for each of the unique patient-treatment incidences, such as for Patient 1-Patient 6, as shown in Table 2.

TABLE 2

| | | | | IVF Phenotype Assignments | | |
|---|---|---|---|---|---|---|
| | | | | | IVF Phenotype #2 | |
| | | | | | Age under 40 + | |
| | IVF Treatment | | | | Oocyte retrieval | IVF Phenotype #3 |
| Unique | Outcomes Dataset | | | IVF Phenotype #1 | cancelled due to | Age 35 to 39 + |
| Patient- | | | IVF | Age under 40 + | too few ovarian | Oocyte retrieval |
| Treatment | IVF | | Treatment | Oocyte retrieval | follicles meeting | cancelled due to |
| Incidences | Treatment | Completed? | Outcome | cancelled | size criteria | OHSS |
| Patient 1 | 1 | Completed | No Live Birth | FALSE | FALSE | FALSE |
| Patient 1 | 2 | Completed | Live Birth | FALSE | FALSE | FALSE |
| Patient 2 | 1 | Completed | No Live Birth | TRUE | FALSE | TRUE |
| Patient 2 | 2 | Completed | Live Birth | FALSE | FALSE | FALSE |
| Patient 3 | 1 | Completed | No Live Birth | TRUE | TRUE | FALSE |
| Patient 3 | 2 | Completed | No Live Birth | FALSE | FALSE | FALSE |
| Patient 4 | 1 | In Progress | Pending | FALSE | FALSE | FALSE |
| Patient 5 | 1 | Completed | No Live Birth | TRUE | TRUE | FALSE |
| Patient 5 | 2 | Completed | Live Birth | FALSE | FALSE | FALSE |
| Patient 6 | 1 | Completed | No Live Birth | TRUE | TRUE | FALSE |
| Patient 6 | 2 | Completed | No Live Birth | TRUE | TRUE | FALSE |

The model is trained to generate model using the at least one IVF phenotype assignment and an IVF outcome for each of the unique patient-treatment incidences. Here, IVF Phenotype #1 is an example of a phenotype assignment; as well as IVF Phenotype #2 and IVF Phenotype #3. These IVF Phenotype assignments use the binary True or False categorical variable.

The model output includes (i) a probability of IVF failure, (ii) a probability of at least one occurring or recurring IVF phenotype, or both

The model training used in this example includes simple mathematical operation of calculating the percentage of cases that would have a certain outcome. Additionally, the model training may use a method selected from the group consisting of logic operations, mathematical operations, techniques in statistics, classification, regression, artificial intelligence, machine learning, generative artificial intelligence learning, natural language processing, unsupervised learning, and supervised learning.

Provided below are examples of model outputs based on the datasets in Tables 1 and 2.

IVF Phenotype #1=TRUE predicts 100% probability of IVF treatment failure for the current IVF treatment.

IVF Phenotype #1=TRUE predicts 50% probability of IVF treatment failure for the next IVF treatment.

IVF Phenotype #1=TRUE is associated with 25% of its own recurrence (recurrence of the same phenotype).

IVF Phenotype #2=TRUE predicts 100% probability of IVF treatment failure for the current IVF treatment.

IVF Phenotype #2-TRUE predicts 33% probability of IVF treatment failure for the next IVF treatment.

IVF Phenotype #2=TRUE is associated with 33% of its own recurrence (recurrence of the same phenotype).

IVF Phenotype #3=TRUE predicts 100% probability of IVF treatment failure for the current IVF treatment.

IVF Phenotype #3=TRUE predicts 0% probability of IVF treatment failure for the next IVF treatment.

IVF Phenotype #3=TRUE is associated with 0% of its own recurrence (recurrence of the same phenotype).

In the computer-implemented method described herein, data for a target patient is applied to the model. For instance, a target patient has completed her 1^{st} IVF treatment (failed, no live birth). The target patient data includes:
(i) at least one intermediate IVF treatment variable:

Was oocyte retrieval cancelled? Yes
If oocyte retrieval is cancelled, what is the reason? too few ovarian follicles meet size criteria
(ii) at least one clinical variable:
Age: 34
(iii) the method assigns at least one IVF phenotype to the target patient:
IVF phenotype #1: TRUE
IVF phenotype #2: TRUE
IVF phenotype #3: FALSE

The computer-implemented method described herein generates a model output for the target patient, where the output can include:
(i) the target patient's probability of IVF failure:
IVF Phenotype #1=TRUE predicts 100% probability of IVF treatment failure for the current IVF treatment.>>Target patient already knows this.
IVF Phenotype #1=TRUE predicts 50% probability of IVF treatment failure for the next IVF treatment.>>This phenotype is not the most specific for this patient.
IVF Phenotype #2-TRUE predicts 100% probability of IVF treatment failure for the current IVF treatment.>>Target patient already knows this.
IVF Phenotype #2=TRUE predicts 33% probability of IVF treatment failure for the next IVF treatment.>>most relevant or personalized to the target patient

The method may provide all the above statements. The method may provide the IVF Phenotype that is more specific for the target patient (more relevant or personalized based on simple logic or additional algorithms). In some embodiments, the method will provide the following output:
IVF Phenotype #2=TRUE predicts 33% probability of IVF treatment failure for the next IVF treatment.

In various embodiments, the output from the model can provide information including:
(ii) the target patient's probability of at least one IVF phenotype that is occurring or recurring:
IVF Phenotype #1=TRUE is associated with 25% of its own recurrence (recurrence of the same phenotype).>>This phenotype is not the most specific for this patient.
IVF Phenotype #2=TRUE is associated with 33% of its own recurrence (recurrence of the same phenotype).>>most relevant or personalized for this patient.

The more relevant, personalized output can provide information including that IVF Phenotype #2=TRUE is associated with 33% of its own recurrence (recurrence of the same phenotype).

According to the exemplary data provided, the model output includes information such as the following: based on comparison with patients with similar clinical profiles and oocyte retrieval cancelled due to poor ovarian response, the probability of failing the next IVF treatment is 33%. There is a 33% chance that oocyte retrieval will be cancelled again for poor ovarian response.

In some instances, the term “probability” is not limited or restricted to an exact or calculated number. In some instances, “Probability” includes a categorical (e.g. low, medium, high) value or numerical ranges (e.g. 0-30%, 30-60%, over 60%).

In some cases, the model output for the above target patient includes:
IVF Phenotype #2=TRUE predicts 25-50% probability of IVF treatment failure for the next IVF treatment.
IVF Phenotype #2=TRUE is associated with 25-50% recurrence

In some embodiments, the model output is presented as a comparison with other patients or in the context of other attributes shared by patients with a similar model output, such as, but not limited to:
20% of patients share the same IVF Phenotype.
25% of patients also have 30% probability of IVF treatment failure.

Half the patients who share the same recurrence rate of IVF Phenotype #2 choose to undergo another IVF treatment.

80% of patients sharing the same IVF phenotype profile choose to do IVF with donor eggs.

### Example 2. Exemplary IVF Phenotype Assignments

In this example, unique patient-treatment incidences (Patient1, Patient2, Patient3, Patient4, Patient5) are analyzed. Patient 1 undergoes a 1^{st} IVF treatment that is completed and results in a “no live birth” outcome. Patient 2 undergoes a 2^{nd} IVF treatment that is completed and results in a “live birth” outcome. The variables of intermediate IVF outcome that are analyzed in the example are (1) the percentage % of retrieved oocytes showing an abnormal morphology, (2) the percentage % of retrieved oocytes progressed to 2PNs, and (3) number of blastocysts. The clinical variables of that are analyzed in the example are age of egg source, AMH level, and clinical diagnosis of PCOS.

TABLE 3

| | | | | Intermediate IVF Treatment | | | | | |
|---|---|---|---|---|---|---|---|---|---|
| | IVF Treatment | | | Outcome Dataset | | | Clinical | | |
| Unique | Outcomes Dataset | | | % retrieved | % retrieved | | Variable Dataset | | |
| Patient- | | | IVF | oocytes showed | oocytes | | | | Clinical |
| Treatment | IVF | | Treatment | abnormal | progressed | number of | Patient | AMH | Diagnosis |
| Incidences | Treatment | Completed? | Outcome | morphology | to 2PNs | blastocysts | Age | (ng/mL) | of PCOS |
| Patient 1 | 1 | Completed | No Live | 10% | 70% | 3 | 33 | 3.5 | Yes |
| | | | Birth | | | | | | |
| Patient 1 | 2 | Completed | Live | 20% | 80% | 4 | 34 | 3.5 | Yes |
| | | | Birth | | | | | | |
| Patient 2 | 1 | Completed | No Live | 70% | 10% | 1 | 38 | 3.6 | Yes |
| | | | Birth | | | | | | |
| Patient 2 | 2 | Completed | Live | 80% | 20% | 2 | 39 | 3.6 | Yes |
| | | | Birth | | | | | | |
| Patient 3 | 1 | Completed | No Live | 60% | 50% | 3 | 32 | 1.6 | No |
| | | | Birth | | | | | | |
| Patient 3 | 2 | Completed | No Live | 55% | 55% | 2 | 32 | 1.6 | No |
| | | | Birth | | | | | | |
| Patient 4 | 1 | In Progress | Pending | pending | pending | pending | 33 | 2.2 | Yes |
| Patient 5 | 1 | Completed | No Live | 15% | 65% | 3 | 33 | 2.8 | No |
| | | | Birth | | | | | | |
| Patient 5 | 2 | Completed | Live | 15% | 45% | 3 | 33 | 2.8 | No |
| | | | Birth | | | | | | |
| Patient 6 | 1 | Completed | No Live | 70% | 15% | 1 | 34 | 0.6 | Yes |
| | | | Birth | | | | | | |
| Patient 6 | 2 | Completed | No Live | 30% | 10% | 1 | 34 | 0.6 | Yes |
| | | | Birth | | | | | | |

The unique patient-treatment incidences (Patient 1, Patient 2, Patient 3, Patient 4, Patient 5) are assigned using the method described herein IVF phenotype assignments which can include a categorical variable or a numerical variable and at least one intermediate IVF treatment outcome or clinical variable. Table 4 shows that an IVF phenotype #1 assignment includes a clinical variable of “Age under 40” and % of retrieved oocytes showing abnormal morphology, where the thresholds are set at <30%, 30-49% or >50%, where the assignment is a categorical variable. The table shows that an IVF phenotype #2 assignment includes a clinical variable of a clinical diagnosis of PCOS and state the % of oocytes progressing to 2PNs, where the assignment is a numerical variable. The table shows that an IVF phenotype #3 assignment includes 3 clinical variables, where the assignment is a True or False variable or value.

TABLE 4

| | | | | IVF Phenotype Assignments | | |
|---|---|---|---|---|---|---|
| | | | | IVF Phenotype #1 | IVF Phenotype #2 | |
| | | | | Age under 40 + % of | Clinical Diagnosis of | IVF Phenotype #4 |
| | | | | retrieved oocytes showing | PCOS + state the % | Age 35 to 39 + PCOS + |
| Unique | IVF Treatment Outcomes Dataset | | | abnormal morphology is <30%, | of oocytes | AMH > 1.0 + 2 or more |
| Patient- | | | IVF | 30-49% or >50% [more than | progressing to | blastocysts [using 3 |
| Treatment | IVF | | Treatment | 2 options possible for | 2PNs [numerical | variables from the |
| Incidences | Treatment | Completed? | Outcome | the categorical variable] | variable] | Clinical variable dataset] |
| Patient 1 | 1 | Completed | No Live | <30% | 70% | FALSE |
| | | | Birth | | | |
| Patient 1 | 2 | Completed | Live Birth | <30% | 80% | FALSE |
| Patient 2 | 1 | Completed | No Live | >50% | 10% | FALSE |
| | | | Birth | | | |
| Patient 2 | 2 | Completed | Live Birth | >50% | 20% | TRUE |
| Patient 3 | 1 | Completed | No Live | >50% | n/a | FALSE |
| | | | Birth | | | |
| Patient 3 | 2 | Completed | No Live | >50% | n/a | FALSE |
| | | | Birth | | | |
| Patient 4 | 1 | In Progress | Pending | pending | pending | pending |
| Patient 5 | 1 | Completed | No Live | <30% | n/a | FALSE |
| | | | Birth | | | |
| Patient 5 | 2 | Completed | Live Birth | <30% | n/a | FALSE |
| Patient 6 | 1 | Completed | No Live | >50% | 15% | FALSE |
| | | | Birth | | | |
| Patient6 | 2 | Completed | No Live | 30-49% | 10% | FALSE |
| | | | Birth | | | |

Table 3 shows an exemplary series of datasets for a group of unique patient-treatment incidences. The method described herein includes inputting data into a phenotype model (also referred to as a first model) described herein including data related to intermediate IVF treatment outcomes variable, data related to clinical variable, and data enabling assignment of at least one IVF phenotype to the individual patient-treatment incidences. The IVF phenotype assignments shown in Table 4 can be a categorical variable including, but not limited to, True or False, or a numerical variable including a continuous variable or a discrete variable.

### Example 3. Exemplary Use of Concordance Model

This example shows the use of a concordance model generated from a plurality of inputs of unique patient-treatment incidences 1001-1030 into the model. The data of the IVF treatment outcomes for each of unique patient-treatment incidences 1001-1030 includes the number of previous IVF treatments (such as 1 previous IVF treatment), if the IVF treatment was completed, and if the IVF outcome was a “live birth” or “no live birth”. The concordance input can be 1 intermediate IVF treatment outcome variable such as the number of blastocysts (see concordance model input #1) or the number of oocytes (see concordance model input #2). See Table 5.

For each unique patient-treatment incidences 1001-1030, the trained concordance model can output a variable relative to, for example, a threshold established by training such that each of the unique patient-treatment incidences 1001-1030 are assigned a number of blastocysts that is either fewer than expected, the same as expected, or greater than expected. See Table 5.

TABLE 5
Unique patient-treatment incidences and a series of datasets.

| | | | | Concordance Model | Concordance Model |
|---|---|---|---|---|---|
| | | | | Input #1 | Input #2 |
| | Unique patient-treatment incidences | | | | |
| | | | | From Intermediate | From Intermediate |
| | IVF Treatment Outcomes Dataset | | | IVF Treatment | IVF Treatment |
| Patient- | | | IVF | Outcome Dataset | Outcome Dataset |
| treatment | IVF | | Treatment | Number of | Number of mature |
| ID number | Treatment | Completed | Outcome | blastocysts | oocytes |
| 1001 | 1 | Completed | No Live Birth | 0 | 10 |
| 1002 | 1 | Completed | Live Birth | 1 | 10 |
| 1003 | 1 | Completed | Live Birth | 4 | 10 |
| 1004 | 1 | Completed | Live Birth | 3 | 10 |
| 1005 | 1 | Completed | No Live Birth | 3 | 10 |
| 1006 | 1 | Completed | No Live Birth | 4 | 10 |
| 1007 | 1 | Completed | Live Birth | 6 | 10 |
| 1008 | 1 | Completed | Live Birth | 5 | 10 |
| 1009 | 1 | Completed | Live Birth | 2 | 10 |
| 1010 | 1 | Completed | No Live Birth | 1 | 10 |
| 1011 | 1 | Completed | No Live Birth | 0 | 5 |
| 1012 | 1 | Completed | Live Birth | 1 | 5 |
| 1013 | 1 | Completed | No Live Birth | 2 | 5 |
| 1014 | 1 | Completed | Live Birth | 3 | 5 |
| 1015 | 1 | Completed | Live Birth | 2 | 5 |
| 1016 | 1 | Completed | No Live Birth | 1 | 5 |
| 1017 | 1 | Completed | No Live Birth | 4 | 5 |
| 1018 | 1 | Completed | Live Birth | 2 | 5 |
| 1019 | 1 | Completed | No Live Birth | 1 | 5 |
| 1020 | 1 | Completed | No Live Birth | 0 | 5 |
| 1021 | 1 | Completed | No Live Birth | 3 | 20 |
| 1022 | 1 | Completed | Live Birth | 1 | 20 |
| 1023 | 1 | Completed | No Live Birth | 6 | 20 |
| 1024 | 1 | Completed | Live Birth | 5 | 20 |
| 1025 | 1 | Completed | No Live Birth | 3 | 20 |
| 1026 | 1 | Completed | Live Birth | 4 | 20 |
| 1027 | 1 | Completed | No Live Birth | 4 | 20 |
| 1028 | 1 | Completed | Live Birth | 6 | 20 |
| 1029 | 1 | Completed | No Live Birth | 3 | 20 |
| 1030 | 1 | Completed | No Live Birth | 2 | 20 |

TABLE 6
Concordance model output (e.g., greater/more than,
same as, fewer/less than).
Is the blastocyst number fewer than, same as or greater
than expected? Use thresholds established by “training”
in Table 5.

Fewer
Fewer
Greater
Same
Same
Greater
Greater
Greater
Same
Fewer
Fewer
Fewer
Same
Greater
Same
Fewer
Greater
Same
Fewer
Fewer
Fewer
Fewer
Greater
Greater
Fewer
Same
Same
Greater
Fewer
Fewer

The concordance model related to the blastocyst number is trained by setting thresholds for the variables of “greater/more than expected”, “same as expected” or “fewer/less than expected”. In the example, the “training” is to use 30 percentile and 80 percentile as thresholds. In other instances, the exact percentile used can vary. Also, other methods of quantifying or comparing can be used, such as from knowledge domain, other logic, statistical or machine learning techniques. Table 7 depicts an exemplary embodiment of training a concordance model.

TABLE 7
Training the concordance model by setting threshold for defining “greater/more
than”, “same as” or “fewer/less than”.

| | | Fewer than expected | | Greater than expected | | |
|---|---|---|---|---|---|---|
| | | using “30% tile | | using “80% tile | | Same as |
| | | inclusive” as threshold | | inclusive” as threshold | | expected |
| Number | | | blastocyst | | blastocyst | blastocyst |
| of mature | | | number meeting | | number meeting | number meeting |
| oocytes | median | threshold | criteria | threshold | criteria | criteria |
| 5 | 1.5 | 1 | 0, 1 | 2.2 | 3, 4, 5, etc. | 2 |
| 10 | 3 | 1.7 | 0, 1 | 4.2 | 4, 5, 6, etc. | 2, 3 |
| 20 | 3.5 | 3 | 0, 1, 2, 3 | 5.2 | 5, 6, 7, etc. | 4 |

The method also includes training the first model to generate first model outputs using at least one concordance model output such as blastocyst number, as shown in Table 8. Probability of IVF failure of the current IVF treatment (or the first model output) is generated according to the concordance model output of Table 7 and its correlation or prediction of IVF treatment failure.

TABLE 8
The first model output showing a probability of IVF failure of the
current treatment that is based on usage of the concordance model
output and its correlation or prediction of IVF treatment failure.

| | Number of | # cases with | | |
|---|---|---|---|---|
| | blastocysts: Fewer | Live Births | Live Birth | Probability |
| | than expected | (LBs) | Rate (LBR) | of IVF Failure |
| 5 | 5 | 1 | 20% | 77% |
| 10 | 3 | 1 | 33% | |
| 20 | 5 | 1 | 20% | |

| | Number of | | | |
|---|---|---|---|---|
| | blastocysts: Greater | # cases | | Probability |
| | than expected | with LBs | LBR | of IVF Failure |
| 5 | 2 | 1 | 50% | 33% |
| 10 | 4 | 3 | 75% | |
| 20 | 3 | 2 | 67% | |
| | Number of | | | |
| | blastocysts: Same as | # cases | | Probability |
| | expected | with LBs | LBR | of IVF Failure |
| 5 | 3 | 2 | 67% | 38% |
| 10 | 3 | 2 | 67% | |
| 20 | 2 | 1 | 50% | |

Table 9 shows data of the IVF treatment outcomes for a first IVF treatment or both a first and second IVF treatment (IVF Treatment No. 1 and IVF Treatment No. 2) for each of unique patient-treatment incidences 1001-1030. For each IVF treatment the concordance model output is assigned “greater/more than expected”, “same as expected” or “fewer/less than expected” blastocyst number, and an IVF outcome variable is a “live birth” or “no live birth”.

TABLE 9
Unique patient-treatment incidences and a series of datasets.

| | Unique patient-treatment incidences | | | |
|---|---|---|---|---|
| | IVF Treatment No. 1 | | | |
| | | Concordance model | IVF Treatment No. 2 | |
| | | output for IVF | | Concordance model output |
| | | Treatment No. 1: Is the | | for IVF Treatment No. 2: Is |
| Patient- | | blastocyst number | | the blastocyst number |
| treatment | IVF Treatment | fewer than, same as or | IVF Treatment | fewer than, same as or |
| ID number | Outcome | greater than expected? | Outcome | greater than expected? |
| 1001 | No Live Birth | Fewer | No Live Birth | Fewer |
| 1002 | Live Birth | Fewer | | |
| 1003 | Live Birth | Greater | | |
| 1004 | Live Birth | Same | | |
| 1005 | No Live Birth | Same | Live Birth | Greater |
| 1006 | No Live Birth | Greater | Live Birth | Same |
| 1007 | Live Birth | Greater | | |
| 1008 | Live Birth | Greater | | |
| 1009 | Live Birth | Same | | |
| 1010 | No Live Birth | Fewer | No Live Birth | Fewer |
| 1011 | No Live Birth | Fewer | No Live Birth | Fewer |
| 1012 | Live Birth | Fewer | | |
| 1013 | No Live Birth | Same | Live Birth | Same |
| 1014 | Live Birth | Greater | | |
| 1015 | Live Birth | Same | | |
| 1016 | No Live Birth | Fewer | No Live Birth | Same |
| 1017 | No Live Birth | Greater | No Live Birth | Same |
| 1018 | Live Birth | Same | | |
| 1019 | No Live Birth | Fewer | Live Birth | Fewer |
| 1020 | No Live Birth | Fewer | No Live Birth | Fewer |
| 1021 | No Live Birth | Fewer | No Live Birth | Fewer |
| 1022 | Live Birth | Fewer | | |
| 1023 | No Live Birth | Greater | No Live Birth | Same |
| 1024 | Live Birth | Greater | | |
| 1025 | No Live Birth | Fewer | Live Birth | Greater |
| 1026 | Live Birth | Same | | |
| 1027 | No Live Birth | Same | No Live Birth | Same |
| 1028 | Live Birth | Greater | | |
| 1029 | No Live Birth | Fewer | Live Birth | Same |
| 1030 | No Live Birth | Fewer | No Live Birth | Fewer |

Using data of Table 9 the first model can generate a first model output representing the probability of a recurring concordance model, such as expected blastocyst number. A probability of IVF failure in a 2^{nd} IVF treatment is correlated or predicted based on an IVF outcome failure in the 1^{st} IVF treatment and the concordance model from the 2^{nd} IVF treatment.

TABLE 10A
The first model output shows the probability of recurrence of concordance model output.

| | | | | Probability of Failure in 2nd IVF |
|---|---|---|---|---|
| Concordance model | | # cases | | Treatment based on: 1) failed 1st |
| output in IVF Treatment | Number | with Live | | treatment and 2) concordance |
| No. 2 after failed 1st IVF | of | Births | Live Birth | model output from 2nd IVF |
| treatment | patients | (LBs) | Rate (LBR) | treatment |
| Blastocyst number is | 7 | 1 | 14% | 86% |
| fewer than expected | | | | |
| Blastocyst number is | 2 | 2 | 100% | 0% |
| greater than expected | | | | |
| Blastocyst number is the | 7 | 3 | 43% | 57% |
| same as expected | | | | |

Data limited to patients who failed a 1^{st} IVF treatment can be analyzed using a concordance model. Patient-treatment incidences of Table 9 fitting such criteria are shown in Table 10B.

TABLE 10B
The first model output showing the probability
of recurrence of concordance model output.

| Concordance | | | |
|---|---|---|---|
| model output in | | | |
| IVF Treatment | | | Probability of |
| No. 1 (limited | | | recurrence of |
| to patients | | # cases | concordance |
| who failed IVF | Number of | with | model |
| Treatment No. 1) | patients | recurrence | output |
| Fewer | 10 | 7 | 70% |
| Same or Greater | 6 | 6 | 100% |

1. A computer-implemented method comprising:
(a) inputting, into a first model, an IVF dataset from at least one group of unique patient-treatment incidences at one or more treatment centers,
wherein the IVF dataset comprises (a) data for generating at least one IVF phenotype assignment for each of the unique patient-treatment incidences, wherein each IVF phenotype assignment is a categorical variable or a numerical variable, (b) IVF outcomes data, (c) intermediate IVF treatment outcomes data, and (d) clinical data,
wherein the IVF outcomes data comprises at least one variable of an IVF outcome, wherein an IVF outcomes variable is an IVF outcomes failure variable, an IVF treatment outcomes variable is an IVF outcomes success variable, or the IVF outcomes variable is an IVF outcomes pending variable,
wherein the intermediate IVF treatment outcomes data comprises a plurality of variables of one or more intermediate IVF treatment outcomes selected from the group consisting of patient oocyte status variables, patient embryo status variables, carrier status variables, and any combination thereof,
wherein the clinical data comprises at least one clinical variable,
wherein the at least one IVF phenotype assignment variable for each unique patient-treatment incidence is based on the at least one intermediate IVF treatment outcomes data variable and the at least one clinical variable; and
(b) training the first model using the at least one IVF phenotype assignment and an IVF outcome variable for each of the unique patient-treatment incidences, thereby generating trained first model outputs for the unique patient-treatment incidences.

2. The method of claim 1, further comprising:
(c) applying, to the trained first model, a target's patient data comprising at least one intermediate IVF treatment variable, at least one clinical variable, and at least one IVF phenotype assigned to the target patient; and
(d) generating a trained first model output for the target patient selected from the group consisting of the target patient's probability of IVF failure, the target patient's probability of at least one occurring or recurring IVF phenotype, and a combination thereof.

3. A computer-implemented method comprising:
(a) inputting, into a first model, an IVF dataset from at least one group of unique patient-treatment incidences at one or more treatment centers,
wherein the IVF dataset comprises (a) data for generating at least one concordance model output of a value of greater or more than expected, same as expected, or fewer or less than expected for each of the unique patient-treatment, (b) IVF outcomes data, (c) intermediate IVF treatment outcomes data, and (d) clinical data;
wherein the IVF outcomes data comprises at least one variable of an IVF treatment outcome, wherein an IVF outcomes variable is an IVF outcomes failure variable, an IVF treatment outcomes variable is an IVF outcomes success variable, or an IVF outcomes variable is an IVF outcomes pending variable;
wherein the intermediate IVF treatment outcomes data comprise a plurality of variables of one or more intermediate IVF treatment outcomes selected from the group consisting of patient oocyte status variables, patient embryo status variables, carrier status variables, and any combination thereof; and
wherein the clinical data comprises at least one clinical variable; and
(b) generating a concordance model output for each of the unique patient-treatment incidences comprising:
(i) inputting, into an untrained concordance model, at least one first concordance input and at least one second concordance input, wherein each first concordance input comprises at least one intermediate IVF treatment outcomes variable or clinical variable; wherein each second concordance input comprises at least one intermediate IVF treatment outcomes or clinical variable;
(ii) training the concordance model to generate a concordance model output; and
(iii) outputting, from the trained concordance model, the concordance model output for each of the unique patient-treatment incidences; and
(c) training the first model to generate trained first model outputs using at least one concordance model output for each of the unique patient-treatment incidences;
wherein the trained first model output comprises one or more selected from the group consisting of (i) a probability of IVF failure, (ii) a probability of at least one occurring or recurring IVF phenotype, and (iii) a probability of at least one occurring or recurring concordance model output.

4. The method of claim 3, further comprising:
(d) applying, to the trained concordance model, a target patient's data comprising (i) the target patient's first concordance input comprising at least one intermediate IVF treatment outcomes variable or clinical variable and (ii) the target patient's second concordance input comprising at least one intermediate IVF treatment outcomes variable or clinical variable,
wherein the at least one intermediate IVF treatment outcomes variable or clinical variable are not identical in the target patient's first and second concordance inputs;
(e) generating the target patient's concordance model output;
(f) applying, to the trained first model, the target patient's concordance model output; and
(g) generating a first model output for the target patient comprising one or more selected from the group consisting of (i) the target patient's probability of IVF failure, (ii) the target patient's probability of at least one occurring or recurring IVF phenotype, and (iii) the target patient's probability of at least one occurring or recurring concordance model output.

5. The method of any one of claims 1-4, wherein the at least one of the IVF outcomes failure variables is selected from the group consisting of: no pregnancy; an undetectable serum B-HCG level of the target patient by 21 days after embryo transfer; a biochemical pregnancy loss after an initially detectable and positive serum B-HCG level declines or lowers to an undetectable levels; a disappearance of a gestational sac; an arrest in fetal development after an earlier detectable stage of fetal development; a diagnosis of a molar pregnancy, and a diagnosis of an ectopic pregnancy.

6. The method of any one of claims 1-5, wherein the IVF outcomes success variable is a live birth.

7. The method of any one of claims 1-6, wherein the at least one IVF outcomes pending variable is selected from the group consisting of (i) at least one planned, intended or contemplated IVF procedure selected from the group consisting of an oocyte retrieval, an in vitro fertilization, an intracytoplasmic sperm injection (ICSI), an embryo culture, an embryo transfer, and an embryo cryopreservation; (ii) occurrence of an embryo transfer but a serum B-HCG test result for the test patient is not available; (iii) at least one planned test selected from the group consisting of a serum B-HCG test, an ultrasound or other imaging test for a gestational sac, an ultrasound or other imaging test for a fetal pole, and an ultrasound or other imaging test for a fetal cardiac activity; (iv) at least one cryopreserved embryo available for transfer; and (v) a variable that is not a variable of the IVF outcome failure or the IVF outcome success.

8. The method of any one of claims 1-7, wherein the patient oocyte status variables are selected from the group consisting of: a cancelled oocyte retrieval procedure due to an inadequate ovarian follicular response based on any criteria; an oocyte retrieval procedure resulting in fewer than expected number of oocytes or no oocytes; an occurrence of premature ovulation; signs or symptoms indicative of ovarian hyperstimulation syndrome (OHSS); 20% or more retrieved oocytes show abnormal morphology or morphology indicative of development arrest such as intact germinal vesicle, immature oocytes, fragmentation, vesicular appearance, granularity, or any other abnormality present in 20% or more of affected oocytes; 20% or more retrieved oocytes have zona pellucida that is thin, thick or absent; and 20% or more retrieved oocytes have abnormalities.

9. The method of any one of claims 1-8, wherein the patient embryo status variables are selected from the group consisting of: a fewer than 2 blastocysts, or euploid blastocysts if preimplantation genetic testing for aneuploidy is performed; at least 20% of embryos at each developmental stage failing to progress to the next developmental stage; at least 20% of blastocysts do not show expansion by day 5, 6, or 7; at least 20% of blastocysts not obtaining a Grade5AA score based on the Gardner score; 20% or more retrieved oocytes failing to develop into mature oocytes, not achieving the 2PN status after in vitro fertilization, not achieving 2PN after ICSI procedure or attempt; at least 20% of blastocysts are not eligible for cryopreservation or uterine transfer; at least 20% of oocytes and embryos at each developmental stage having one or more detectable abnormal features; at least 20% of oocytes and/or embryos at each developmental stage having an accelerated or delayed timing to progress to the next developmental stage; at least 20% of oocytes and/or embryos having an overall accelerated or delayed progression of two more developmental stages; at least 20% of oocytes and/or embryos being cultured in an abnormal in vitro culture media or environment; at least 20% of embryos at each stage are not progressing to the next stage; at least 20% of oocytes and embryos derived from an abnormal oocyte, sperm, embryo, or an abnormal oocyte-sperm interaction; at least 20% of oocytes and/or embryos having an abnormal level of at least one nucleic acid transcript; at least 20% of oocytes and/or embryos having an abnormal level of at least one polypeptide; at least 20% of oocytes and/or embryos having an abnormal level or structure of at least one intracellular or secreted molecule, analyte, cellular or gene product; and at least 20% of oocytes and/or embryos having at least one gene variant associated with or causes or confers risk for IVF failure, occurrence or recurrence of at least one IVF phenotype, pregnancy loss, recurrent pregnancy loss, fetal developmental defects, congenital birth defects, newborn illnesses.

10. The method of any one of claims 1-9, wherein the carrier status variables are selected from the group consisting of: a less than expected or less than 7 mm thickness of an intended gestational carrier's endometrial lining within 14 days of an embryo transfer; a less than expected or less than 7 mm thickness of an intended gestational carrier's endometrial lining within 48 hours of an embryo transfer; an inadequate thickness of an intended gestational carrier's endometrial lining resulting in a cancelled oocyte retrieval, a cancelled embryo transfer or a freezing of embryos; an endometrium abnormality; an endometrial cavity fluid or mass; and an abnormal appearance or abnormality of the female reproductive tract.

11. The method of any one of claims 1-10, wherein the at least one clinical variable is selected from the group consisting of: a clinical variable selected from the group consisting of body mass index (BMI), relative distances between any 2 or more facial features, ratio of upper to lower body, relative length of certain body part, abdominal girth, body fat density, and muscle mass of an oocyte source, sperm source, and/or gestational carrier, and qualitative biometrics, quantitative biometrics, and other biometrics of an oocyte source, sperm source, and/or gestational carrier; an ovarian function variable selected from the group consisting of serum anti-mullerian hormone (AMH) levels, day 3 follicle stimulating hormone (D3 FSH) levels, antral follical count (AFC), serum progesterone levels, serum inhibin levels, biochemical analysis or gene expression of granulosa cells or follicular fluid, premature ovarian failure, ovarian failure, medical conditions associated with decreased ovarian function or premature ovarian failure, exposure to medicines or substances harmful to ovarian function; an IVF treatment protocol variable selected from the group consisting of IVF treatment protocol, type of medication, dosage, dosage dose schedule and adjustments, the use of ovarian suppression medication, gonadotropin medication, oral contraceptive pill, gonadotropin releasing hormone (GnRH) agonists, GnRH antagonists, luteinizing hormone (LH), human chorionic gonadotropin (HCG), progesterone or any progestin type, estradiol or any estrogen type, clomiphene, letrozole, recombinant hormones, purified hormones, long-acting, in vitro maturation of oocytes, in vitro gametogenesis; a demographic variable selected from the group consisting of age, race, ethnicity, social determinants of health, health insurance coverage, IVF coverage, household income, education level, type of employment; a reproductive history variable selected from the group consisting of number of months or years of conception attempts, a same-sex couple, a heterosexual couple, a single woman, a history of pregnancy, a number or live birth pregnancy loss; a number of past fertility treatments and outcomes, and a type of past fertility treatments and outcomes; a reproductive tract variable for the oocyte source, sperm source and/or gestational carrier; a diagnostic test variable selected from the group consisting of endocrine diagnostic tests, liver function, renal function, electrolyte, cholesterol panel, imaging, metabolic panel, autoimmune disease panel, ovarian function, coagulation, recurrent pregnancy loss panel, prenatal panel, male endocrine panel, ovarian function or suppression diagnostic tests for gestational carrier or patient, and any diagnostic tests indicating the patient's organ function, any measure of nutrients or metabolites such as folate, any folate metabolites; a clinical diagnosis variable related to a reproductive or sexual function selected from the group consisting of: tubal disease, recurrent pregnancy loss, endometriosis, uterine fibroid, congenital abnormalities affecting reproductive function, polycystic ovaries, polycystic ovarian syndrome, anovulation, irregular menstrual cycles, menorrhagia, ovarian cyst, ovarian masses, cervical abnormalities, diseases of the hypothalamus or pituitary glands or other hormonal dysfunction affecting reproductive functions, male factor, abnormalities in semen analysis, obstructive azoospermia, non-obstructive azoospermia, vas deferens abnormalities, erectile dysfunction, ejaculatory dysfunction, any gene variants associated with or causes or confers risk for reproductive dysfunction, clinical infertility, IVF failure, occurrence or recurrence of at least one IVF phenotype, pregnancy loss, recurrent pregnancy loss, obstetrical complications, fetal developmental defects, congenital birth defects, newborn illnesses; a clinical diagnosis variable related to a medical condition not related to a reproductive or sexual function; a microbiology variable affecting the oocyte source, sperm source and/or gestational carrier, selected from the group consisting of a variable relating to an infection with a known or unknown pathogen, an abnormal microbiome, an imbalance of normal flora, infection or relative levels of lactobacillus, gardnerella, Atopobium vaginae, megasphera, candida species or any micro-organisms, and a mix of micro-organisms causing or supporting abnormal pH levels in the reproductive tract; a sperm abnormality variable selected from the group consisting of an abnormal sperm count, abnormal count of motile sperm, abnormal percentage of sperm having certain motility measures, abnormal percentage or count of sperm having abnormal morphology, abnormal percentage or count of sperm having abnormal function, abnormal percentage or count of sperm having an abnormal amount of DNA, organelles, subcellular structures or any other detectable abnormal features; and a gene variant variable related to a clinical variable, demographic variable, reproductive history variable, reproductive tract variable, microbiology variable, microbiome variable, laboratory test variable, or clinical diagnosis variable for the oocyte source, the sperm source and/or the gestational carrier, or a sperm abnormality variable.

12. The method of any one of claims 1-11, wherein the first model output is for the target patient's current or future IVF treatment.

13. The method of any one of claims 1-12, further comprising applying, to the trained first model, at least one IVF treatment outcomes variable from the target patient for a current IVF treatment.

14. The method of any one of claims 1-13, further comprises applying, to the trained first model, at least one IVF treatment outcomes variable from the target patient for one or more past IVF treatments.

15. The method of any one of claims 1-14, wherein the first model output further provides one or more selected from the group consisting of a level of correlation or predictive relationship between two IVF phenotypes assigned to the target patient, and a level of correlation or predictive relationship between the target patient's concordance model output and the target patient's probability of an IVF failure.

16. The method of any one of claims 1-2 and 5-15, wherein the at least one IVF phenotype assignment is a categorical variable selected from the group consisting of true or false; semi-quantitative categories indicating disease severity; and non-quantitative categories.

17. The method of any one of claims 1-2 and 5-15, wherein the at least one IVF phenotype assignment is a numerical variable and wherein the numerical variable is a continuous variable quantifying from least to most severe.

18. The method of any one of claims 1-2 and 5-15, wherein the at least one IVF phenotype assignment is a numerical variable and wherein the numerical variable is a discrete variable.

19. The method of any one of claims 3-15, wherein the at least one concordance model output assignment for each for the unique patient-treatment incidences utilizes set thresholds in a quantitative scale or prognostic score thresholds based on a statistical comparison or a composite prognostic score generated by clinical outcomes prediction models.

20. The method of any one of claims 3-15 and 18-19, wherein a plurality of performance metrics of the trained concordance model are compared to those of a control model (i) having no concordance model inputs and relying on mathematical averages for performance metrics, if the concordance model input uses one intermediate IVF outcome or clinical variable; (ii) having only one variable as a concordance model input, if the concordance model input uses two intermediate IVF outcome or clinical variables; or (iii) having fewer variables than the concordance model input, if the concordance model input uses more than two intermediate IVF outcome or clinical variables.

21. The method of any one of the preceding claims, further comprising evaluating a plurality of performance metrics of at least the trained first model or the trained concordance model using any method selected from the group consisting of AUC of an ROC curve, posterior log-likelihood, any log-likelihood, dynamic range, reclassification, calibration, precision, recall, and F1 score.

22. The method of claim 21, wherein the evaluating the plurality of performance metrics utilizes an independent dataset that is not used in training.

23. The method of claim 21, wherein at least one of the plurality of performance metrics of the trained first model is compared to a corresponding performance metric of a control first model, wherein the control first model utilizes no input variables and outputs a mathematical average; and wherein the control first model comprises only one or fewer input variables than the trained first model.

24. The method of any one of the preceding claims, wherein the training comprises utilizing a method selected from the group consisting of logic operations, mathematical operations, techniques in statistics, classification, regression, artificial intelligence, machine learning, generative artificial intelligence learning, natural language processing, unsupervised learning, supervised learning and any methods for building a prediction model, an algorithm, or an explainer.

25. The method of any one of the preceding claims, further comprising executing the computer-executed method and performing an assay method for detecting at least one gene variant variable correlating with one or more selected from the group consisting of the IVF outcome, the intermediate IVF outcome, and the IVF phenotype in the target patient.

26. The method of any one of the preceding claims, further comprising executing the computer-executed method and analyzing the at least one gene variant variable of the target patient and utilizing the gene variant variable in the inputting step (a).

27. The method of claim 26 or 27, wherein the at least one gene variant variable is selected from the group consisting of a single nucleotide polymorphism (SNP), copy number variation (CNV), DNA insertion, DNA deletion, DNA duplication, DNA inversion, structural chromosomal abnormality, DNA methylation, nuclear DNA variant, and mitochondrial DNA variant, familial gene variant, de novo gene variant, polygenic gene variant, haplotype, and variant of a chromosomal region.

28. A diagnostic test for a target patient comprising executing the computer-implemented method of any one of the preceding claims for the target patient.

29. A method of providing a clinician a probability of an IVF outcome occurring or recurring of a current or prospective IVF treatment for a target patient comprising: executing the computer-implemented method of any one of claims 1-28 for the target patient; and displaying on a device a user interface showing the probability of the IVF outcome for the target patient.

30. A computer-implemented method for identifying and using diagnostic gene variants associated with or predictive for IVF outcomes comprising:
(a) inputting, into a gene variant model, an IVF dataset from at least one group of unique patient-treatment incidences at one or more treatment centers,
wherein the dataset comprises (i) data for generating at least one IVF phenotype assignment and at least one gene variant assignment, wherein the at least one IVF phenotype assignment is a discrete variable or a continuous variable, and wherein the at least one gene variant assignment is a categorical variable or a numerical variable; (ii) IVF outcomes data; (iii) intermediate IVF treatment outcomes data; (iv) clinical data; and (v) gene variant data;
wherein the IVF outcomes data comprises at least one variable of an IVF outcome, wherein the IVF outcomes variable is an IVF outcomes failure variable, the IVF treatment outcomes variable is an IVF outcomes success variable, or the IVF outcomes variable is an IVF outcomes pending variable;
wherein the intermediate IVF treatment outcomes data comprise a plurality of variables of one or more intermediate IVF treatment outcomes selected from the group consisting of patient oocyte status variables, patient embryo status variables, carrier status variables, and any combination thereof;
wherein the clinical data comprises at least one clinical variable;
wherein the gene variant data comprises at least one gene variant variable of an oocyte source, sperm source, and/or a gestational carrier; and
(b) training the gene variant model using the at least one IVF phenotype assignment for each of the unique patient-treatment incidences and the at least one gene variant variable, thereby generating gene variant model outputs for each of the unique patient-treatment incidences, wherein each gene variant model output comprises one or more selected from the group consisting of (i) a probability of IVF failure and (ii) a probability of at least one occurring or recurring IVF phenotype;
(c) applying, to the trained gene variant model, a target patient's data comprising: at least one intermediate IVF treatment variable, at least one clinical variable, at least one IVF phenotype assigned to the target patient, and at least one gene variant variable for the target patient; and
(d) generating a trained gene variant model output for the target patient selected from the group consisting of the target patient's probability of IVF failure, the target patient's probability of at least one IVF phenotype occurring or recurring, and a combination thereof.

31. The method of claim 30, wherein the at least one gene variant variable is selected from the group consisting of a single nucleotide polymorphism (SNP), copy number variation (CNV), DNA insertion, DNA deletion, DNA duplication, DNA inversion, structural chromosomal abnormality, DNA methylation, nuclear DNA variant, and mitochondrial DNA variant, familial gene variant, de novo gene variant, polygenic gene variant, haplotype, and variant of a chromosomal region.

32. The method of any one of claims 26-27 and 29-31, wherein the structural chromosomal abnormality is selected from the group consisting of chromosomal insertion, chromosomal deletion, chromosomal duplication, chromosomal inversion, balanced translocation, and Robertsonian translocation.

33. The method of any one of claims 26-27 and 29-32, wherein the gene variant is classified based on functional effect selected from the group consisting of normal function, likely normal function, functional variance of unknown significance, hypothetical function effect, likely functional effect, hypomorphic allele, functional effect, loss of function, and gain of function.

34. The method of any one of claims 26-27 and 29-33, wherein the gene variant is classified based on a clinical importance selected from the group consisting of clinical variant of unknown significance, right match for phenotype, known risk factor, possible risk factor, variant-of-interest, pathogenic variant, penetrance-graded when known, pathogenic with high penetrance, and pathogenic with moderate penetrance.

35. The method of any one of claims 26-27 and 29-34, wherein the gene variant is associated with, causes or confers risk for abnormal quality or function of uterine implantation, ovary, oocyte, embryo, sperm, fetal development and function, neonatal health and function, congenital anomalies, newborn illnesses, or adult-onset disease.

36. The method of any one of claims 26-27 and 29-35, wherein the at least one gene variant is identified by a method selected from the group consisting of GWAS, family pedigree, animal models, in vitro cell or tissue studies, studies utilizing genetic editing technology or related technology, case-control studies, whole genome sequencing, exome sequencing, SNP microarray, PCR, CGH array, utilizing gene variant databases and/or genetic ancestry algorithms or results, and any combination thereof.

37. The method of any one of claims 26-27 and 29-36, wherein the at least one gene variant is a potential pathogenic gene variant indicating a treatment option and/or a reproductive outcome prognosis, wherein the treatment options is selected from the group consisting of IVF, ICSI, in vitro maturation of oocytes, in vitro gametogenesis, use of alternative sperm source, oocyte source, embryo source or uterine source, use of a genetic editing technology, use of genetic analysis of oocytes, embryos or other cell types, and any combination thereof.
